(19) United States
(12) Patent Application Publication
(10) Pub. No.: US 2024/0165045 A1
(43) Pub. Date: May 23, 2024

(54) DRY POWDER FORMULATIONS OF NUCLEIC ACID LIPID NANOPARTICLES

(71) Applicant: BOARD OF REGENTS, THE UNIVERSITY OF TEXAS SYSTEM, Austin, TX (US)

(72) Inventors: Robert O. WILLIAMS, III, Austin, TX (US); Chaeho MOON, Austin, TX (US); Sawittree SAHAKIJPIJARN, Austin, TX (US); Haiyue XU, Austin, TX (US); Zhengrong CUI, Austin, TX (US)

(21) Appl. No.: 18/549,645

(22) PCT Filed: Mar. 8, 2022
(86) PCT No.: PCT/US22/19318

§ 371 Date: Sep. 8, 2023

Related U.S. Application Data
(60) Provisional application No. 63/158,280, filed on Mar. 8, 2021.

(51) Int. Cl. A61K 9/51; A61K 39/215

(52) U.S. Cl. CPC A61K 9/5123; A61K 9/5192; A61K 39/215; A61K 2039/53

(57) ABSTRACT

In some aspects, the present disclosure provides dry powder pharmaceutical compositions comprising: (A) a nucleic acid; (B) a lipid nanoparticle; wherein the nucleic acid is substantially encapsulated in the lipid nanoparticle; (C) a sugar; and (D) a pharmaceutically acceptable polymer. The dry powder composition may show improved stability relative to solution based pharmaceutical composition of nucleic acid therapeutic agents encapsulated in a lipid nanoparticle.

This application claims the benefit of priority to U.S. Provisional Application No. 63/158,280, filed on Mar. 8, 2021, the entire contents of which are hereby incorporated by reference.

### BACKGROUND

### 1. Field

The present disclosure relates generally to the field of pharmaceuticals and pharmaceutical manufacture. More particularly, it concerns compositions and methods of preparing a pharmaceutical composition comprising lipid nanoparticles containing nucleic acids as dry powders.

### 2. Description of Related Art

Nucleic acids therapeutics are a growing field of pharmaceuticals with the number of new nucleic acid drugs increasing year over year. Unfortunately, these compounds require the use of specific formulations in order for them to be therapeutically effective in vivo. In particular, these compositions are generally formulated as lipid nanoparticles as an aqueous solution. These formulations often require specific storage requirements. Two of the SARS CoV2 vaccines, which are both based upon mRNA, are formulated as lipid nanoparticles. These compositions have well established and lengthy preparation, storage, and transport protocols because of the cold chain required to maintain the activity of the vaccine. Some of the cold chain requirements include keeping the vaccine at a temperature of —80° until it is about to be used and then must be used shortly after. This particular requirement necessitates the installation of supply chain that can maintain this temperature preventing rapid expansion into the general population in the developed countries as well as severely limiting the access of these compositions into underdeveloped countries. Therefore, there remains a desire to develop pharmaceutical compositions that show improved stability.

### SUMMARY OF THE INVENTION

In some aspects, the present disclosure provides dry powder pharmaceutical compositions of lipid nanoparticle encapsulated nucleic acid therapeutics that show improved stability. In some embodiments, the present disclosure provides pharmaceutical compositions comprising:
(A) a nucleic acid;
(B) one or more lipids sufficient to form a lipid nanoparticle;
(C) a sugar; and
(D) a pharmaceutically acceptable polymer;
   wherein the pharmaceutical composition is formulated as a powder and wherein the nucleic acid is substantially encapsulated in the lipid nanoparticle.

In some embodiments, the powder is a dry powder. In some embodiments, the powder is free of any water. In some embodiments, the powder is substantially free of any water. In some embodiments, the powder is essentially free of any water.

In some embodiments, the nucleic acid is an mRNA such as an mRNA that encodes for an antigen. In some embodiments, the antigen is an anti-viral antigen such as a SARS CoV2 antigen. In some embodiments, the SARS CoV2 antigen is an mRNA which encodes for the SARS CoV2 spike protein or a modified version thereof. In some embodiments, the mRNA encodes for a modified version of the SARS CoV2 spike protein. In some embodiments, the modified version of the SARS CoV2 spike protein contains one or more proline substitutions. In some embodiments, the nucleic acid contains one or more modifications. In some embodiments, the nucleic acid comprises one, two, three, four, five, six, seven, eight, nine, or ten modifications. In some embodiments, the modifications comprise a 5′ cap, one or more untranslated regions, a signal peptide, a linker sequence, a poly(A) tail, a modified nucleotide, or a series of the same nucleotides. In some embodiments, the modifications include a 5′ untranslated region or a 3′ untranslated region.

In some embodiments, the pharmaceutical compositions comprise one, two, three, four, five, six, seven, or eight lipids. In some embodiments, the pharmaceutical compositions comprise three, four, or five lipids. In some embodiments, the pharmaceutical compositions comprise a first lipid. In some embodiments, the first lipid is a lipid with two or more hydrophobic groups.

In some embodiments, the pharmaceutical compositions comprise a second lipid. In some embodiments, the second lipid is a phospholipid. In some embodiments, the phospholipid is phosphocholine. In some embodiments, the phospholipid is distearoylphosphatidylcholine (DSPC).

In some embodiments, the pharmaceutical compositions comprise a third lipid. In some embodiments, the third lipid is a PEGylated lipid. In some embodiments, the PEGylated lipid comprises one or more polyethylene glycol units. In some embodiments, the PEGylated lipid comprises a polyethylene glycol unit with a molecular weight from about 1 kilodalton to about 10 kilodaltons. In some embodiments, the PEGylated lipid is N,N-dimyristylamide of 2-hydroxyacetic acid with a polyethylene glycol unit with a molecular weight of about 2 kilodaltons. In some embodiments, the PEGylated lipid is N,N-ditetradecylacetamide with a polyethylene glycol unit with a molecular weight of about 2 kilodaltons.

In some embodiments, the pharmaceutical compositions comprise a fourth lipid such as a steroid. In some embodiments, the steroid is a sterol. In some embodiments, the sterol is cholesterol. In some embodiments, the first, second, third, and fourth lipids form a lipid nanoparticle. In some embodiments, the nucleic acid is essentially encapsulated in the lipid nanoparticle. In some embodiments, the nucleic acid is entirely encapsulated in the lipid nanoparticle.

In some embodiments, the sugar is a polysaccharide such as a disaccharide or a trisaccharide. In some embodiments, the polysaccharide is a disaccharide. In some embodiments, the disaccharide contains a glucose. In some embodiments, the disaccharide is sucrose or trehalose. In some embodiments, the disaccharide is sucrose. In other embodiments, the sugar is a sugar alcohol. In some embodiments, the sugar alcohol is sorbitol, xylitol, or mannitol.

In some embodiments, the pharmaceutically acceptable polymer is a copolymer. In some embodiments, the copolymer is a triblock copolymer. In some embodiments, the copolymer comprises one or more polyoxypropylene units and one or more polyoxyethylene units. In some embodiments, the copolymer comprises one polyoxypropylene unit and two polyoxyethylene units. In some embodiments, the copolymer comprises a polyoxypropylene unit with a polyoxyethylene unit on each side of the polyoxypropylene unit. In some embodiments, the polyoxypropylene unit has a molecular weight from about 500 g/mol to about 5000 g/mol. In some embodiments, the molecular weight of the polyoxypropylene unit is from about 750 g/mol to about 3000 g/mol. In some embodiments, the molecular weight of the polyoxypropylene unit is from about 1500 g/mol to about 2000 g/mol. In some embodiments, the molecular weight of the polyoxypropylene unit is about 1800 g/mol. In some embodiments, each of the polyoxyethylene unit has a molecular weight from about 100 g/mol to about 2500 g/mol. In some embodiments, the molecular weight of the polyoxyethylene unit is from about 250 g/mol to about 2000 g/mol. In some embodiments, the molecular weight of the polyoxyethylene unit is from about 600 g/mol to about 1000 g/mol. In some embodiments, the molecular weight of the polyoxyethylene unit is about 800 g/mol. In some embodiments, the pharmaceutically acceptable polymer is poloxamer P188.

In some embodiments, the pharmaceutical compositions further comprise one or more salts. In some embodiments, the salt is a phosphate buffer. In other embodiments, the salt is sodium chloride. In other embodiments, the salt is the solids content from phosphate buffered saline (PBS).

In some embodiments, the pharmaceutical compositions comprise from about 0.05% w/w to about 50% w/w of the lipid nanoparticles. In some embodiments, the pharmaceutical compositions comprise from 0.1% w/w to about 50% w/w of the lipid nanoparticles. In some embodiments, the pharmaceutical compositions comprise from 0.25% w/w to about 50% w/w of the lipid nanoparticles. In some embodiments, the pharmaceutical compositions comprise from about 1% w/w to about 15% w/w of the lipid nanoparticles. In some embodiments, the pharmaceutical compositions comprise from about 2% w/w to about 10% w/w of the lipid nanoparticles. In some embodiments, the pharmaceutical compositions comprise from about 2% w/w to about 5% w/w of the lipid nanoparticles. In some embodiments, the pharmaceutical compositions comprise from about 6% w/w to about 10% w/w of the lipid nanoparticles.

In some embodiments, the pharmaceutical compositions comprise from about 25% w/w to about 98% w/w of the sugar. In some embodiments, the pharmaceutical compositions comprise from about 40% w/w to about 95% w/w of the sugar. In some embodiments, the pharmaceutical compositions comprise from about 50% w/w to about 90% w/w of the sugar. In some embodiments, the pharmaceutical compositions comprise from about 50% w/w to about 70% w/w of the sugar. In some embodiments, the pharmaceutical compositions comprise from about 80% w/w to about 90% w/w of the sugar.

In some embodiments, the pharmaceutical compositions comprise from about 0.1% w/w to about 25% w/w of the pharmaceutically acceptable polymer. In some embodiments, the pharmaceutical composition comprises from about 0.25% w/w to about 15% w/w of the pharmaceutically acceptable polymer. In some embodiments, the pharmaceutical compositions comprise from about 0.4% w/w to about 5% w/w of the pharmaceutically acceptable polymer. In some embodiments, the pharmaceutical compositions comprise from about 0.5% w/w to about 2.5% w/w of the pharmaceutically acceptable polymer. In some embodiments, the pharmaceutical compositions comprise from about 1.0% w/w to about 1.5% w/w of the pharmaceutically acceptable polymer.

In some embodiments, the pharmaceutical compositions comprise from about 1% w/w to about 50% w/w of each salt. In some embodiments, the pharmaceutical compositions comprise from about 2% w/w to about 45% w/w of each salt. In some embodiments, the pharmaceutical compositions comprise from about 2.5% w/w to about 40% w/w of each salt. In some embodiments, the pharmaceutical compositions comprise from about 5% w/w to about 30% w/w of each salt. In some embodiments, the pharmaceutical compositions comprise from about 5% w/w to about 10% w/w of each salt. In some embodiments, the pharmaceutical compositions comprise from about 20% w/w to about 30% w/w of each salt.

In some embodiments, the pharmaceutical compositions comprise one or more particles. In some embodiments, each of the particles comprise the lipid nanoparticles, the pharmaceutically acceptable polymer, and the sugar. In some embodiments, the mRNA recovery after processing is greater than 60%. In some embodiments, the mRNA recovery is greater than 70%. In some embodiments, the mRNA recovery is greater than 80%. In some embodiments, the lipid nanoparticles have a Z-average is from about 50 nm to about 250 nm. In some embodiments, the Z-average is from about 75 nm to about 200 nm. In some embodiments, the Z-average is from about 80 nm to about 150 nm.

In some embodiments, the pharmaceutical compositions shows less than 10% degradation after 1 month when stored at a temperature below 30° C. In some embodiments, the pharmaceutical compositions showed less than 5% degradation after 1 month. In some embodiments, the pharmaceutical compositions showed less than 3% degradation after 1 month. In some embodiments, the pharmaceutical compositions showed less than 1% degradation after 1 month. In some embodiments, the pharmaceutical compositions showed reduced degradation when stored below 5° C. In some embodiments, the pharmaceutical compositions have a low bulk density.

In some embodiments, the pharmaceutical compositions has been reconstituted into a solution. In some embodiments, the solution is made with water. In some embodiments, the solution is made with phosphate buffered saline. In some embodiments, the solution is made with citrate buffer. In some embodiments, the pharmaceutical compositions further comprise one or more additional excipients. In some embodiments, the additional excipient is a protein, an amino acid, a second pharmaceutically acceptable polymer, an antioxidant, or a surfactant. In some embodiments, the pharmaceutical compositions comprise:
(A) a nucleic acid; wherein the nucleic acid is an mRNA that encodes for a SARS CoV2 antigen; wherein the SARS CoV2 antigen is a modified version of the SARS CoV2 spike protein;
(B) one or more lipids sufficient to form a lipid nanoparticle; wherein the lipid nanoparticle comprises a first, second, third, and fourth lipid; wherein the first lipid is ionizable lipid, the second lipid is a phospholipid, the third lipid is a PEGylated lipid, and the fourth lipid is a sterol;
(C) a sugar, wherein the sugar is a disaccharide; and
(D) a pharmaceutically acceptable polymer; wherein the pharmaceutically acceptable polymer is a triblock copolymer with two polyoxyethylene units and one polyoxypropylene unit.

In another aspect, the present disclosure provides methods of preparing a pharmaceutical composition described herein comprising:
(A) dissolving a mixture of lipid nanoparticle encapsulating a nucleic acid, a sugar, and a pharmaceutically acceptable polymer in a solvent to obtain a pharmaceutical mixture;
(B) applying the pharmaceutical mixture to a surface at a surface temperature below 0° C. to obtain a frozen pharmaceutical mixture; and
(C) collecting the frozen pharmaceutical mixture and drying the frozen pharmaceutical mixture to obtain a pharmaceutical composition.

In some embodiments, the solvent is water. In some embodiments, the pharmaceutical mixture further comprises a second solvent. In some embodiments, the second solvent is an organic solvent. In some embodiments, the organic solvent is acetonitrile, tert-butanol, or 1,4-dioxane. In some embodiments, the methods further comprise admixing the mixture with a salt. In some embodiments, the first solvent is mixed with the second solvent to obtain a homogenous pharmaceutical mixture. In some embodiments, the pharmaceutical mixture is admixed until the pharmaceutical mixture is clear.

In some embodiments, the pharmaceutical mixture comprises a solid content from about 0.05% w/v to about 25% w/v of the mixture. In some embodiments, the solid content is from about 0.1% w/v to about 10% w/v of the mixture. In some embodiments, the solid content is from about 0.15% w/v to about 5% w/v of the mixture. In some embodiments, the solid content is from about 0.2% w/v to about 2.5% w/v of the mixture. In some embodiments, the solid content is from about 0.5% w/v to about 1.25% w/v of the mixture.

In some embodiments, the pharmaceutical mixture is applied at a feed rate from about 0.5 mL/min to about 5 mL/min. In some embodiments, the feed rate is from about 1 mL/min to about 3 mL/min In some embodiments, the feed rate is about 2 mL/min. In some embodiments, the pharmaceutical mixture is applied with a nozzle such as a needle. In some embodiments, the pharmaceutical mixture is applied from a height from about 2 cm to about 50 cm. In some embodiments, the height is from about 5 cm to about 20 cm. In some embodiments, the height is about 10 cm.

In some embodiments, the surface temperature is from about 0° C. to −190° C. In some embodiments, the surface temperature is from about −25° C. to about −125° C. In some embodiments, the surface temperature is about −100° C. In some embodiments, the surface is a rotating surface. In some embodiments, the surface is rotating at a speed from about 5 rpm to about 500 rpm. In some embodiments, the surface is rotating at a speed from about 100 rpm to about 400 rpm. In some embodiments, the surface is rotating at a speed of about 200 rpm. In other embodiments, the surface is stationary.

In some embodiments, the frozen pharmaceutical composition is dried by lyophilization. In some embodiments, the frozen pharmaceutical composition is dried at a first reduced pressure. In some embodiments, the first reduced pressure is from about 10 mTorr to 500 mTorr. In some embodiments, the first reduced pressure is from about 50 mTorr to about 250 mTorr. In some embodiments, the first reduced pressure is about 100 mTorr. In some embodiments, the frozen pharmaceutical composition is dried at a first reduced temperature. In some embodiments, the first reduced temperature is from about 0° C. to −100° C. In some embodiments, the first reduced temperature is from about −20° C. to about −60° C. In some embodiments, the first reduced temperature is about −40° C. In some embodiments, the frozen pharmaceutical composition is dried for a primary drying time period from about 3 hours to about 36 hours. In some embodiments, the primary drying time period is from about 6 hours to about 24 hours. In some embodiments, the primary drying time period is about 20 hours.

In some embodiments, the frozen pharmaceutical composition is dried a secondary drying time period. In some embodiments, the frozen pharmaceutical composition is dried a secondary drying time at a second reduced pressure. In some embodiments, the secondary drying time is at a reduced pressure is from about 10 mTorr to 500 mTorr. In some embodiments, the secondary drying time is at a reduced pressure is from about 50 mTorr to about 250 mTorr. In some embodiments, the secondary drying time is at a reduced pressure is about 100 mTorr. In some embodiments, the frozen pharmaceutical composition is dried a secondary drying time at a second reduced temperature. In some embodiments, the second reduced temperature is from about 0° C. to 30° C. In some embodiments, the second reduced temperature is from about 10° C. to about 30° C. In some embodiments, the second reduced temperature is about 25° C. In some embodiments, the frozen pharmaceutical composition is dried for a second time for a second time period from about 0.5 hours to about 36 hours. In some embodiments, the second time period is from about 6 hours to about 24 hours. In some embodiments, the second time period is about 20 hours.

In some embodiments, the temperature is changed from the first reduced temperature to the second reduced temperature over a ramping time period. In some embodiments, the ramping time period is from about 1 hours to about 36 hours. In some embodiments, the ramping time period is from about 6 hours to about 24 hours. In some embodiments, the ramping time period is about 20 hours.

In still yet another aspect, the present disclosure provides pharmaceutical composition prepared using the methods described herein.

In still another aspect, the present disclosure provides methods of preventing a disease or disorder comprising administering to a patient in need thereof a therapeutically effective amount of a pharmaceutical composition described herein.

In yet another aspect, the present disclosure provides methods of treating a disease or disorder comprising administering to a patient in need thereof a therapeutically effective amount of a pharmaceutical composition described herein.

In some embodiments, the disease or disorder is an infection of a virus. In some embodiments, the virus is SARS CoV2. In some embodiments, the methods reduce the severity of the infection. In some embodiments, the methods prevent the patient from developing a symptomatic infection.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows a representative transmission electron microscopic (TEM) image of mRNA-LNPs after they were subjected to thin-film freeze-drying (i.e. formulation 5) and reconstitution.

### DESCRIPTION OF ILLUSTRATIVE EMBODIMENTS

In some aspects, the present disclosure relates to dry powder pharmaceutical compositions of lipid nanoparticle encapsulated the therapeutic nucleic acid. These dry powder compositions may have one or more benefits over traditional lipid nanoparticle solutions. For example, these dry powder pharmaceutical compositions may show improved stability or eliminate the use of a cold chain. Details of these compositions are provided in more detail below.

### I. Pharmaceutical Compositions

In some aspects, the present disclosure provides pharmaceutical composition as dry powders. These dry powder pharmaceutical compositions may be stored and at a later date reconstituted into an injectable solution with the addition of a solvent such as water, saline, or phosphate buffered saline. These solvent systems may further comprise a sugar such as sucrose or dextrose. Within the dry powder, the pharmaceutical composition comprises a nucleic acid encapsulated within a lipid nanoparticle, a sugar molecule, and a pharmaceutically acceptable polymer. The compositions may further comprise one or more salts.

In some embodiments, the pharmaceutical compositions have one or more lipid nanoparticles that encapsulate the nucleic acid. These lipid nanoparticles may have a Z-average from 25 nm to about 500 nm, 50 nm to about 250 nm, 75 nm to about 200 nm, or from about 80 nm to about 150 nm. In some embodiments, the Z-average is from about 25 nm, 30 nm, 40 nm, 50 nm, 60 nm, 70 nm, 80 nm, 90 nm, 100 nm, 110 nm, 120 nm, 125 nm, 130 nm, 140 nm, 150 nm, 160 nm, 170 nm, 175 nm, 180 nm, 190 nm, 200 nm, 225 nm, to about 250 nm, or any range derivable therein. The dry powder may be stored at a temperature below 40° C., 35° C., 30° C., 25° C., 20° C., 15° C., 10° C., 5° C., 0° C., or −5° C. When stored at these temperature, the pharmaceutical composition may show less degradation. In some embodiments, the degradation may be measured by changes in the activity of the vaccine. In other embodiments, the degradation is measured by changes in the Z-average. The pharmaceutical composition may show less than 50%, 40%, 30%, 25%, 20%, 15%, 10%, 7.5%, 5%, 3%, 2%, or 1% degradation after a period of time. This degradation may be measured over a time period of 1 week, 2 weeks, 3 weeks, 1 month, 2 months, 3 months, 4 months, 6 months, 9 months or 1 year. Furthermore, after preparing the pharmaceutical composition, the amount of mRNA that is recovered may be 50%, 55%, 60%, 65%, 70%, 75%, 80%, 85%, 90%, 92.5%, 95%, 96%, 97%, 98%, or 99% of the starting amount of mRNA used.

Additionally, the pharmaceutical composition may exhibit a low bulk density. The bulk density may be 0.040±0.003 g/mL. Furthermore, the bulk density of the pharmaceutical composition may be viewed in relation to its specific surface area. The pharmaceutical composition may have a specific surface area from about 5 m²/g to about 1,000 m²/g, from about 7.5 m²/g to about 500 m²/g, from about 10 m²/g to about 250 m²/g, from about 12.5 m²/g to about 100 m²/g, or from about 15 m²/g to about 75 m²/g. The specific surface area of the pharmaceutical composition may be greater than 5 m²/g, 6 m²/g, 7 m²/g, 7.5 m²/g, 8 m²/g, 9 m²/g, 10 m²/g, 12.5 m²/g, 15 m²/g, or 17.5 m²/g. The specific surface area of the pharmaceutical composition may be from about 5 m²/g, 6 m²/g, 7 m²/g, 7.5 m²/g, 8 m²/g, 9 m²/g, 10 m²/g, 12.5 m²/g, 15 m²/g, 17.5 m²/g, 20 m²/g, 22.5 m²/g, 25 m ²/g, 30 m²/g, 40 m²/g, 50 m²/g, 60 m²/g, 70 m²/g, 75 m²/g, 80 m²/g, 90 m²/g, 100 m²/g, 250 m²/g, 500 m²/g, to about 1,000 m²/g, or any range derivable therein The specific surface area may be determined by the single-point Braummer-Emmett-Teller (BET) method using a Monosorb rapid surface area analyzer.

### A. Nucleic Acids

“Nucleic acid,” “nucleic acid sequence,” “oligonucleotide,” “polynucleotide” or other grammatical equivalents as used herein means at least two nucleotides, either deoxyribonucleotides or ribonucleotides, or analogs thereof, covalently linked together. Polynucleotides are polymers of any length, including, e.g., 20, 50, 100, 200, 300, 500, 1000, 2000, 3000, 5000, 7000, 10,000, etc. A polynucleotide described herein generally contains phosphodiester bonds, although in some cases, nucleic acid analogs are included that may have at least one different linkage, e.g., phosphoramidate, phosphorothioate, phosphorodithioate, or O-methylphophoroamidite linkages, and peptide nucleic acid backbones and linkages. Mixtures of naturally occurring polynucleotides and analogs can be made; alternatively, mixtures of different polynucleotide analogs, and mixtures of naturally occurring polynucleotides and analogs may be made. The following are non-limiting examples of polynucleotides: a gene or gene fragment, exons, introns, messenger RNA (mRNA), transfer RNA, ribosomal RNA, ribozymes, cDNA, cRNA, recombinant polynucleotides, branched polynucleotides, plasmids, minicircle DNA, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, the term polynucleotide encompasses both the double-stranded form and each of two complementary single-stranded forms known or predicted to make up the double-stranded form. A polynucleotide is composed of a specific sequence of four nucleotide bases: adenine (A), cytosine (C), guanine (G), thymine (T), and uracil (U) for thymine when the polynucleotide is RNA. Thus, the term “polynucleotide sequence” is the alphabetical representation of a polynucleotide molecule. Unless otherwise indicated, a particular polynucleotide sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

Modified RNA contemplates certain chemical modifications that confer increased stability and low immunogenicity to mRNAs, thereby facilitating expression of therapeutically important proteins. For instance, N1-methyl-pseudouridine (N1mΨ) outperforms several other nucleoside modifications and their combinations in terms of translation capacity. In some embodiments, the nucleic acids used herein may have the uracils replaced with psuedouracils such as 1-methyl-3′-pseudouridylyl bases. In some embodiments, some of the uracils are replaced, but in other embodiments, all of the uracils have been replaced. In addition to turning off the immune/eIF2α phosphorylation-dependent inhibition of translation, incorporated N1mΨ nucleotides dramatically alter the dynamics of the translation process by increasing ribosome pausing and density on the mRNA. Increased ribosome loading of modified mRNAs renders them more permissive for initiation by favoring either ribosome recycling on the same mRNA or de novo ribosome recruitment. Such modifications could be used to enhance antibody expression in vivo following inoculation with RNA. The RNA, whether native or modified, may be delivered as naked RNA or in a delivery vehicle, such as a lipid nanoparticle.

Each of these are described in more detail below.

### 1. Nucleic Acids

In some aspects of the present disclosure, the lipid nanoparticles comprise one or more nucleic acids. In some embodiments, the lipid nanoparticles comprises one or more nucleic acids present in a weight ratio to the lipid nanoparticles from about 5:1 to about 1:100. In some embodiments, the weight ratio of nucleic acid to lipid nanoparticles is from about 5:1, 2.5:1, 1:1, 1:5, 1:10, 1:15, 1:20, 1:25, 1:30, 1:35, 1:40, 1:45, 1:50, 1:60, 1:70, 1:80, 1:90, or 1:100, or any range derivable therein. In some embodiments, the weight ratio is about 1:40. In addition, it should be clear that the present disclosure is not limited to the specific nucleic acids disclosed herein. The present disclosure is not limited in scope to any particular source, sequence, or type of nucleic acid, however, as one of ordinary skill in the art could readily identify related homologs in various other sources of the nucleic acid including nucleic acids from non-human species (e.g., mouse, rat, rabbit, dog, monkey, gibbon, chimp, ape, baboon, cow, pig, horse, sheep, cat and other species). It is contemplated that the nucleic acid used in the present disclosure can comprises a sequence based upon a naturally-occurring sequence. Allowing for the degeneracy of the genetic code, sequences that have at least about 50%, usually at least about 60%, more usually about 70%, most usually about 80%, preferably at least about 90% and most preferably about 95% of nucleotides that are identical to the nucleotide sequence of the naturally-occurring sequence. In another embodiment, the nucleic acid is a complementary sequence to a naturally occurring sequence, or complementary to 75%, 80%, 85%, 90%, 95% and 100%. Longer polynucleotides encoding 250, 500, 1000, 1212, 1500, 2000, 2500, 3000 or longer are contemplated herein.

The nucleic acid used herein may be derived from genomic DNA, i.e., cloned directly from the genome of a particular organism. In preferred embodiments, however, the nucleic acid would comprise complementary DNA (cDNA). Also contemplated is a cDNA plus a natural intron or an intron derived from another gene; such engineered molecules are sometime referred to as “mini-genes.” At a minimum, these and other nucleic acids of the present disclosure may be used as molecular weight standards in, for example, gel electrophoresis.

The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

In some embodiments, the nucleic acid comprises one or more antisense segments which inhibits expression of a gene or gene product. Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

### 2. Modified Nucleobases

In some embodiments, the nucleic acids of the present disclosure comprise one or more modified nucleosides comprising a modified sugar moiety. Such compounds comprising one or more sugar-modified nucleosides may have desirable properties, such as enhanced nuclease stability or increased binding affinity with a target nucleic acid relative to an oligonucleotide comprising only nucleosides comprising naturally occurring sugar moieties. In some embodiments, modified sugar moieties are substituted sugar moieties. In some embodiments, modified sugar moieties are sugar surrogates. Such sugar surrogates may comprise one or more substitutions corresponding to those of substituted sugar moieties.

In some embodiments, modified sugar moieties are substituted sugar moieties comprising one or more non-bridging sugar substituent, including but not limited to substituents at the 2′ and/or 5′ positions. Examples of sugar substituents suitable for the 2′-position, include, but are not limited to: 2′-F, 2′-OCH₃ (“OMe” or “O-methyl”), and 2′-O(CH₂)₂OCH₃ (“MOE”). In certain embodiments, sugar substituents at the 2′ position is selected from allyl, amino, azido, thio, O-allyl, O—C₁-C₁₀ alkyl, O—C₁-C₁₀ substituted alkyl; OCF₃, O(CH₂)₂SCH₃, O(CH₂)₂-O—N(Rm)(Rn), and O—CH₂—C(═O)—N(Rm)(Rn), where each Rm and Rn is, independently, H or substituted or unsubstituted C₁-C₁₀ alkyl. Examples of sugar substituents at the 5′-position, include, but are not limited to: 5′-methyl (R or S); 5′-vinyl, and 5′-methoxy. In some embodiments, substituted sugars comprise more than one non-bridging sugar substituent, for example, T-F-5′-methyl sugar moieties (see, e.g., PCT International Application WO 2008/101157, for additional 5′,2′-bis substituted sugar moieties and nucleosides).

Nucleosides comprising 2′-substituted sugar moieties are referred to as 2′-substituted nucleosides. In some embodiments, a 2′-substituted nucleoside comprises a 2′-substituent group selected from halo, allyl, amino, azido, SH, CN, OCN, CF₃, OCF₃, O, S, or N(Rₘ)-alkyl; O, S, or N(Rₘ)-alkenyl; O, S or N(Rₘ)-alkynyl; O-alkylenyl-O-alkyl, alkynyl, alkaryl, aralkyl, O-alkaryl, O-aralkyl, O(CH₂)₂SCH₃, O(CH₂)₂—O—N(Rₘ)(Rₙ) or O—CH₂—C(═O)—N(Rₘ)(Rₙ), where each Rₘ and Rₙ is, independently, H, an amino protecting group or substituted or unsubstituted C₁-C₁₀ alkyl. These 2′-substituent groups can be further substituted with one or more substituent groups independently selected from hydroxyl, amino, alkoxy, carboxy, benzyl, phenyl, nitro (NO₂), thiol, thioalkoxy (S-alkyl), halogen, alkyl, aryl, alkenyl and alkynyl.

In some embodiments, a 2′-substituted nucleoside comprises a 2′-substituent group selected from F, NH₂, N₃, OCF₃, O—CH₃, O(CH₂)₃NH₂, CH₂—CH═CH₂, O—CH₂—CH═CH₂, OCH₂CH₂OCH₃, O(CH₂)₂SCH₃, O—(CH₂)₂—O—N(Rₘ)(Rₙ), O(CH₂)₂O(CH₂)₂N(CH₃)₂, and N-substituted acetamide (O—CH₂—C(═O)—N(Rₘ)(Rₙ) where each Rₘ and Rₙ is, independently, H, an amino protecting group or substituted or unsubstituted C₁-C₁₀ alkyl.

In some embodiments, a 2′-substituted nucleoside comprises a sugar moiety comprising a 2′-substituent group selected from F, OCF₃, O—CH₃, OCH₂CH₂OCH₃, O(CH₂)₂SCH₃, O(CH₂)₂—O—N(CH₃)₂, —O(CH₂)₂O(CH₂)₂N(CH₃)₂, and O—CH₂—C(═O)—N(H)CH₃.

In some embodiments, a 2′-substituted nucleoside comprises a sugar moiety comprising a 2′-substituent group selected from F, O—CH₃, and OCH₂CH₂OCH₃.

Certain modified sugar moieties comprise a bridging sugar substituent that forms a second ring resulting in a bicyclic sugar moiety. In some such embodiments, the bicyclic sugar moiety comprises a bridge between the 4′ and the 2′ furanose ring atoms. Examples of such 4′ to 2′ sugar substituents, include, but are not limited to: —[C(Rₐ)(R_{b})]ₙ—, —[C(Rₐ)(R_{b})]ₙ—O—, —C(RₐR_{b})—N(R)—O— or, —C(RₐR_{b})—O—N(R)—; 4′-CH₂-2′, 4′-(CH₂)₂-2′, 4′-(CH₂)—O—2′ (LNA); 4′-(CH₂)13 S-2′; 4′-(CH₂)₂—O-2′ (ENA); 4′-CH(CH₃)—O-2′ (cEt) and 4′-CH(CH₂OCH₃)—O-2′, and analogs thereof (see, e.g., U.S. Pat. No. 7,399,845); 4′-C(CH₃)(CH₃)—O-2′ and analogs thereof, (see, e.g., WO 2009/006478); 4′-CH₂—N(OCH₃)-2′ and analogs thereof (see, e.g., WO2008/150729); 4′-CH₂—O—N(CH₃)-2′ (see, e.g., US2004/0171570, published Sep. 2, 2004); 4′-CH₂—O—N(R)-2′, and 4′-CH₂—N(R)—O-2′-, wherein each R is, independently, H, a protecting group, or C₁-C₁₂ alkyl; 4′-CH₂—N(R)—O-2′, wherein R is H, C₁-C₁₂ alkyl, or a protecting group (see, U.S. Pat. No. 7,427,672); 4′-CH₂—C(H)(CH₃)-2′ (see, e. g. , Chattopadhyaya et al. , J. Org. Chem., 2009, 74, 118-134); and 4′-CH₂—C(═CH₂)-2′ and analogs thereof (see, PCT International Application WO 2008/154401).

In some embodiments, such 4′ to 2′ bridges independently comprise from 1 to 4 linked groups independently selected from —[C(Rₐ)(R_{b})]ₙ—, —C(Rₐ)═C(R_{b})—, —C(Rₐ)═N—, —C(═NRₐ)—, —C(═O)—, —C(═S)—, —Si(Rₐ)₂—, —S(═O)ₓ—, and —N(Rₐ)—; wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, LJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Nucleosides comprising bicyclic sugar moieties are referred to as bicyclic nucleosides or BNAs. Bicyclic nucleosides include, but are not limited to, (A) α-L-Methyleneoxy (4′-CH₂—O-2′) BNA, (B) β-D-Methyleneoxy (4′-CH₂—O-2′) BNA (also referred to as locked nucleic acid or LNA), (C) Ethyleneoxy (4′-(CH₂)₂—O-2′) BNA, (D) Aminooxy (4′-CH₂—O—N(R)-2′) BNA, (E) Oxyamino (4′-CH₂—N(R)—O-2′) BNA, (F) Methyl(methyleneoxy) (4′-CH(CH₃)—O-2′) BNA (also referred to as constrained ethyl or cEt), (G) methylene-thio (4′-CH₂—S-2′) BNA, (H) methylene-amino (4′-CH2-N(R)-2′) BNA, (I) methyl carbocyclic (4′-CH₂—CH(CH₃)-2′) BNA, (J) propylene carbocyclic (4′-(CH and (K) Methoxy(ethyleneoxy) (4′-CH(CH₂OMe)-O-2′) BNA (also referred to as constrained MOE or cMOE).

Additional bicyclic sugar moieties are known in the art, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 129(26) 8362-8379 (Jul. 4, 2007); Elayadi et al., Curr. Opinion Invens. Drugs, 2001, 2, 5561; Braasch et al., Chem. Biol., 2001, 8, 1-7; Orum et al., Curr. Opinion Mol. Ther., 2001, 3, 239-243; U.S. Pat. Nos. 7,053,207, 6,268,490, 6,770,748, 6,794,499, 7,034,133, 6,525,191, 6,670,461, and 7,399,845; WO 2004/106356, WO 1994/14226, WO 2005/021570, and WO 2007/134181; U.S. Patent Publication Nos. US 2004/0171570, US 2007/0287831, and US 2008/0039618; U.S. Ser. Nos. 12/129,154, 60/989,574, 61/026,995, 61/026,998, 61/056,564, 61/086,231, 61/097,787, and 61/099,844; and PCT International Applications Nos. PCT/US2008/064591, PCT/US2008/066154, and PCT/US2008/068922.

In some embodiments, bicyclic sugar moieties and nucleosides incorporating such bicyclic sugar moieties are further defined by isomeric configuration. For example, a nucleoside comprising a 4′-2′ methylene-oxy bridge, may be in the .alpha.-L configuration or in the .beta.-D configuration. Previously, α-L-methyleneoxy (4′-CH₂—O-2′) bicyclic nucleosides have been incorporated into antisense oligonucleotides that showed antisense activity (Frieden et al., Nucleic Acids Research, 2003, 21, 6365-6372).

In some embodiments, substituted sugar moieties comprise one or more non-bridging sugar substituent and one or more bridging sugar substituent (e.g., 5′-substituted and 4′-2′ bridged sugars; PCT International Application WO 2007/134181, wherein LNA is substituted with, for example, a 5′-methyl or a 5′-vinyl group).

In some embodiments, modified sugar moieties are sugar surrogates. In some such embodiments, the oxygen atom of the naturally occurring sugar is substituted, e.g., with a sulfer, carbon or nitrogen atom. In some such embodiments, such modified sugar moiety also comprises bridging and/or non-bridging substituents as described above. For example, certain sugar surrogates comprise a 4′-sulfur atom and a substitution at the 2′-position (see, e.g., published U.S. Patent Application US 2005/0130923) and/or the 5′ position. By way of additional example, carbocyclic bicyclic nucleosides having a 4′-2′ bridge have been described (see, e.g., Freier et al., Nucleic Acids Research, 1997, 25(22), 4429-4443 and Albaek et al., J. Org. Chem., 2006, 71, 7731-7740).

In some embodiments, sugar surrogates comprise rings having other than 5-atoms. For example, in some embodiments, a sugar surrogate comprises a six-membered tetrahydropyran. Such tetrahydropyrans may be further modified or substituted. Nucleosides comprising such modified tetrahydropyrans include, but are not limited to, hexitol nucleic acid (HNA), anitol nucleic acid (ANA), manitol nucleic acid (MNA) (see Leumann, C J. Bioorg. & Med. Chem. (2002) 10:841-854), and fluoro HNA (F-HNA).

In some embodiments, the modified THP nucleosides of Formula VII are provided wherein q₁, q₂, q₃, q₄, q₅, q₆ and q₇ are each H. In certain embodiments, at least one of q₁, q₂, q₃, q₄, q₅, q₆ and q₇ is other than H. In some embodiments, at least one of q₁, q₂, q₃, q₄, q₅, q₆ and q₇ is methyl. In some embodiments, THP nucleosides of Formula VII are provided wherein one of R₁ and R₂ is F. In certain embodiments, R₁ is fluoro and R₂ is H, R₁ is methoxy and R₂ is H, and R₁ is methoxyethoxy and R₂ is H.

Many other bicyclo and tricyclo sugar surrogate ring systems are also known in the art that can be used to modify nucleosides for incorporation into antisense 15 compounds (see, e.g., review article: Leumann, J. C, Bioorganic & Medicinal Chemistry, 2002, 10, 841-854).

Combinations of modifications are also provided without limitation, such as 2′-F-5′-methyl substituted nucleosides (see PCT International Application WO 2008/101157 for other disclosed 5′,2′-bis substituted nucleosides) and replacement of the ribosyl ring oxygen atom with S and further substitution at the 2′-position (see U.S. Patent Publication US 2005/0130923) or alternatively 5′-substitution of a bicyclic nucleic acid (see PCT International Application WO 2007/134181 wherein a 4′-CH₂—O-2′ bicyclic nucleoside is further substituted at the 5′ position with a 5′-methyl or a 5′-vinyl group). The synthesis and preparation of carbocyclic bicyclic nucleosides along with their oligomerization and biochemical studies have also been described (see, e.g., Srivastava et al., 2007).

In some embodiments, the present disclosure provides oligonucleotides comprising modified nucleosides. Those modified nucleotides may include modified sugars, modified nucleobases, and/or modified linkages. The specific modifications are selected such that the resulting oligonucleotides possess desirable characteristics. In some embodiments, oligonucleotides comprise one or more RNA-like nucleosides. In some embodiments, oligonucleotides comprise one or more DNA-like nucleotides.

In some embodiments, nucleosides of the present disclosure comprise one or more unmodified nucleobases. In certain embodiments, nucleosides of the present disclosure comprise one or more modified nucleobases.

In some embodiments, modified nucleobases are selected from: universal bases, hydrophobic bases, promiscuous bases, size-expanded bases, and fluorinated bases as defined herein. 5-substituted pyrimidines, 6-azapyrimidines and N-2, N-6 and O-6 substituted purines, including 2-aminopropyladenine, 5-propynyluracil; 5-propynylcytosine; 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 15 7-methyladenine, 2-F-adenine, 2-amino-adenine, 8-azaguanine and 8-azaadenine, 7-deazaguanine and 7-deazaadenine, 3-deazaguanine and 3-deazaadenine, universal bases, hydrophobic bases, promiscuous bases, size-expanded bases, and fluorinated bases as defined herein. Further modified nucleobases include tricyclic pyrimidines such as phenoxazine cytidine([5,4-b][1,4]benzoxazin-2(3H)-one), phenothiazine cytidine (1H-pyrimido [5,4-b][1,4]benzothiazin-2(3H)-one), G-clamps such as a substituted phenoxazine cytidine (e.g., 9-(2-aminoethoxy)-H-pyrimido[5,4-13][1,4]benzoxazin-2(3H)-one), carbazole cytidine (²H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, Kroschwitz, J. I., Ed., John Wiley & Sons, 1990, 858-859; those disclosed by Englisch et al., 1991; and those disclosed by Sanghvi, Y. S., 1993.

Representative United States Patents that teach the preparation of certain of the above noted modified nucleobases as well as other modified nucleobases include without limitation, U.S. Pat. Nos. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653 and 6,005,096, each of which is herein incorporated by reference in its entirety.

In some embodiments, the present disclosure provides oligonucleotides comprising linked nucleosides. In such embodiments, nucleosides may be linked together using any internucleoside linkage. The two main classes of internucleoside linking groups are defined by the presence or absence of a phosphorus atom. Representative phosphorus containing internucleoside linkages include, but are not limited to, phosphodiesters (P═O), phosphotriesters, methylphosphonates, phosphoramidate, and phosphorothioates (P═S). Representative non-phosphorus containing internucleoside linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Modified linkages, compared to natural phosphodiester linkages, can be used to alter, typically increase, nuclease resistance of the oligonucleotide. In some embodiments, internucleoside linkages having a chiral atom can be prepared as a racemic mixture, or as separate enantiomers. Representative chiral linkages include, but are not limited to, alkylphosphonates and phosphorothioates. Methods of preparation of phosphorous-containing and non-phosphorous-containing internucleoside linkages are well known to those skilled in the art.

The oligonucleotides described herein contain one or more asymmetric centers and thus give rise to enantiomers, diastereomers, and other stereoisomeric configurations that may be defined, in terms of absolute stereochemistry, as (R) or (S), α or β such as for sugar anomers, or as (D) or (L) such as for amino acids etc. Included in the antisense compounds provided herein are all such possible isomers, as well as their racemic and optically pure forms.

Neutral internucleoside linkages include without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

Additional modifications may also be made at other positions on the oligonucleotide, particularly the 3′ position of the sugar on the 3′ terminal nucleotide and the 5′ position of 5′ terminal nucleotide. For example, one additional modification of the ligand conjugated oligonucleotides of the present disclosure involves chemically linking to the oligonucleotide one or more additional non-ligand moieties or conjugates which enhance the activity, cellular distribution or cellular uptake of the oligonucleotide. Such moieties include but are not limited to lipid moieties such as a cholesterol moiety (Letsinger et al., 1989), cholic acid (Manoharan et al., 1994), a thioether, e.g., hexyl-5-tritylthiol (Manoharan et al., 1992; Manoharan et al., 1993), a thiocholesterol (Oberhauser et al., 1992), an aliphatic chain, e.g., dodecandiol or undecyl residues (Saison-Behmoaras et al., 1991; Kabanov et al., 1990; Svinarchuk et al., 1993), a phospholipid, e.g., di-hexadecyl-rac-glycerol or triethylammonium 1,2-di-O-hexadecyl-rac-glycero-3-H-phosphonate (Manoharan et al., 1995; Shea et al., 1990), a polyamine or a polyethylene glycol chain (Manoharan et al., 1995), or adamantane acetic acid (Manoharan et al., 1995), a palmityl moiety (Mishra et al., 1995), or an octadecylamine or hexylamino-carbonyl-oxycholesterol moiety (Crooke et al., 1996).

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

### B. Lipid Nanoparticles

In some embodiments, the lipid nanoparticles used herein may contain one, two, three, four, five, six, seven, eight, nine, or ten lipids. These lipids may include triglycerides, phospholipids, steroids or sterols, a PEGylated lipids, or a group with a ionizable group such as an alkyl amine and one or more hydrophobic groups such as C6 or greater alkyl groups.

### 3. Steroids and Steroid Derivatives

In some aspects of the present disclosure, the lipid nanoparticles are mixed with one or more steroid or a steroid derivative. In some embodiments, the steroid or steroid derivative comprises any steroid or steroid derivative. As used herein, in some embodiments, the term “steroid” is a class of compounds with a four ring 17 carbon cyclic structure which can further comprises one or more substitutions including alkyl groups, alkoxy groups, hydroxy groups, oxo groups, acyl groups, or a double bond between two or more carbon atoms. In one aspect, the ring structure of a steroid comprises three fused cyclohexyl rings and a fused cyclopentyl ring as shown in the formula below:

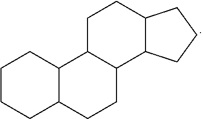 [SMILES: C1CCC2C(C1)CCC1C3CCCC3CCC21]

In some embodiments, a steroid derivative comprises the ring structure above with one or more non-alkyl substitutions. In some embodiments, the steroid or steroid derivative is a sterol wherein the formula is further defined as:

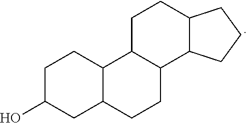 [SMILES: OC1CCC2C(CCC3C4CCCC4CCC23)C1]

In some embodiments of the present disclosure, the steroid or steroid derivative is a cholestane or cholestane derivative. In a cholestane, the ring structure is further defined by the formula:

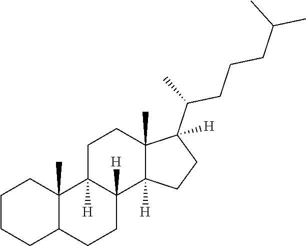

As described above, a cholestane derivative includes one or more non-alkyl substitution of the above ring system. In some embodiments, the cholestane or cholestane derivative is a cholestene or cholestene derivative or a sterol or a sterol derivative. In other embodiments, the cholestane or cholestane derivative is both a cholestere and a sterol or a derivative thereof.

### 4. PEG or PEGylated Lipid

In some aspects of the present disclosure, the lipid nanoparticles are mixed with one or more PEGylated lipids (or PEG lipid). In some embodiments, the present disclosure comprises using any lipid to which a PEG group has been attached. In some embodiments, the PEG lipid is a diglyceride which also comprises a PEG chain attached to the glycerol group. In other embodiments, the PEG lipid is a compound which contains one or more C6-C24 long chain alkyl or alkenyl group or a C6-C24 fatty acid group attached to a linker group with a PEG chain. Some non-limiting examples of a PEG lipid includes a PEG modified phosphatidylethanolamine and phosphatidic acid, a PEG ceramide conjugated, PEG modified dialkylamines and PEG modified 1,2-diacyloxypropan-3-amines, PEG modified diacylglycerols and dialkylglycerols. In some embodiments, PEG modified diastearoylphosphatidylethanolamine or PEG modified dimyristoyl-sn-glycerol. In some embodiments, the PEG modification is measured by the molecular weight of PEG component of the lipid. In some embodiments, the PEG modification has a molecular weight from about 100 to about 15,000. In some embodiments, the molecular weight is from about 200 to about 500, from about 400 to about 5,000, from about 500 to about 3,000, or from about 1,200 to about 3,000. The molecular weight of the PEG modification is from about 100, 200, 400, 500, 600, 800, 1,000, 1,250, 1,500, 1,750, 2,000, 2,250, 2,500, 2,750, 3,000, 3,500, 4,000, 4,500, 5,000, 6,000, 7,000, 8,000, 9,000, 10,000, 12,500, to about 15,000. Some non-limiting examples of lipids that may be used in the present disclosure are taught by U.S. Pat. No. 5,820,873, WO 2010/141069, or U.S. Pat. No. 8,450,298, which is incorporated herein by reference.

### 5. Phospholipid

In some aspects of the present disclosure, the lipid nanoparticles are mixed with one or more phospholipids. In some embodiments, any lipid which also comprises a phosphate group. In some embodiments, the phospholipid is a structure which contains one or two long chain C6-C24 alkyl or alkenyl groups, a glycerol or a sphingosine, one or two phosphate groups, and, optionally, a small organic molecule. In some embodiments, the small organic molecule is an amino acid, a sugar, or an amino substituted alkoxy group, such as choline or ethanolamine In some embodiments, the phospholipid is a phosphatidylcholine. In some embodiments, the phospholipid is distearoylphosphatidylcholine or dioleoylphosphatidylethanolamine. In some embodiments, other zwitterionic lipids are used, where zwitterionic lipid defines lipid and lipid-like molecules with both a positive charge and a negative charge.

### 6. Ionizable Lipids

In some aspects of the present disclosure, lipid nanoparticle containing compounds containing lipophilic and cationic components, wherein the cationic component is ionizable, are provided. In some embodiments, the cationic ionizable lipids contain one or more groups which is protonated at physiological pH but may deprotonated and has no charge at a pH above 8, 9, 10, 11, or 12. The ionizable cationic group may contain one or more protonatable amines which are able to form a cationic group at physiological pH. The cationic ionizable lipid compound may also further comprise one or more lipid components such as two or more fatty acids with C₆-C₂₄ alkyl or alkenyl carbon groups. These lipid groups may be attached through an ester linkage or may be further added through a Michael addition to a sulfur atom. In some embodiments, these compounds may be a dendrimer, a dendron, a polymer, or a combination thereof.

In some aspects of the present disclosure, composition containing compounds containing lipophilic and cationic components, wherein the cationic component is ionizable, are provided. In some embodiments, ionizable cationic lipids refer to lipid and lipid- like molecules with nitrogen atoms that can acquire charge (pKa). These lipids may be known in the literature as cationic lipids. These molecules with amino groups typically have between 2 and 6 hydrophobic chains, often alkyl or alkenyl such as C6-C24 alkyl or alkenyl groups, but may have at least 1 or more that 6 tails.

In some embodiments, the amount of the lipid nanoparticle with the nucleic acid encapsulated in the pharmaceutical composition is from about 0.1% w/w to about 50% w/w, from about 0.25% w/w to about 25% w/w, from about 0.5% w/w to about 20% w/w, from about 1% w/w to about 15% w/w, from about 2% w/w to about 10% w/w, from about 2% w/w to about 5% w/w, or from about 6% w/w to about 10% w/w. In some embodiments, the amount of the lipid nanoparticle with the nucleic acid encapsulated in the pharmaceutical composition is from about 0.1% w/w, 0.25% w/w, 0.5% w/w, 1% w/w, 2.5% w/w, 5% w/w, 7.5% w/w, 10% w/w, 15% w/w, 20% w/w, 25% w/w, 30% w/w, 35% w/w, 40% w/w, 45% w/w, 50% w/w, 55% w/w, 60% w/w, 65% w/w, 70% w/w, 75% w/w, 80% w/w, 85% w/w, 90% w/w, to about 95% w/w, or any range derivable therein.

### C. Sugar

In some aspects, the present disclosure comprises one or more sugars formulated into pharmaceutical compositions. In some embodiments, the sugars used herein are saccharides. These saccharides may be used to act as a lyoprotectant that protects the pharmaceutical composition from destabilization during the drying process. These water-soluble excipients include carbohydrates or saccharides such as disaccharides such as sucrose, trehalose, or lactose, a trisaccharide such as fructose, glucose, galactose comprising raffinose, polysaccharides such as starches or cellulose, or a sugar alcohol such as xylitol, sorbitol, or mannitol. In some embodiments, these excipients are solid at room temperature. Some non- limiting examples of sugar alcohols include erythritol, threitol, arabitol, xylitol, ribitol, mannitol, sorbitol, galactitol, fucitol, iditol, inositol, volemitol, isomalt, maltitol, lactitol, maltotritol, maltotetraitol, or a polyglycitol.

In some embodiments, the amount of the sugar in the pharmaceutical composition is from about 25% w/w to about 98% w/w, 40% w/w to about 95% w/w, 50% w/w to about 90% w/w, 50% w/w to about 70% w/w, or from about 80% w/w to about 90% w/w. In some embodiments, the amount of the sugar in the pharmaceutical composition is from about 10% w/w, 15% w/w, 20% w/w, 25% w/w, 30% w/w, 35% w/w, 40% w/w, 45% w/w, 50% w/w, 52.5% w/w, 55% w/w, 57.5% w/w, 60% w/w, 62.5% w/w, 65% w/w, 67.5% w/w, 70% w/w, 75% w/w, 80% w/w, 82.5% w/w, 85% w/w, 87.5% w/w, 90% w/w, to about 95% w/w, or any range derivable therein.

### D. Pharmaceutically Acceptable Polymer

In some embodiments, the pharmaceutically acceptable polymer is a copolymer. The pharmaceutically acceptable polymer may further comprise one, two, three, four, five, or six subunits of discrete different types of polymer subunits. These polymer subunits may include polyoxypropylene, polyoxyethylene, or a similar subunit. In particular, the pharmaceutically acceptable polymer may comprise at least one hydrophobic subunit and at least one hydrophilic subunit. In particular, the copolymer may have hydrophilic subunits on each side of a hydrophobic unit. The copolymer may have a hydrophilic subunit that is polyoxyethylene and a hydrophobic subunit that is polyoxypropylene.

Each of the polyoxyethylene subunits have a molecular weight from about 50 g/mol to about 5000 g/mol, from about 100 g/mol to about 2500 g/mol, from about 250 g/mol to about 2000 g/mol, from about 500 g/mol to about 1500 g/mol, from about 600 g/mol to about 1000 g/mol, or from about 700 g/mol to about 900 g/mol. The polyoxyethylene subunits may have a subunit from about 50 g/mol, 100 g/mol, 250 g/mol, 500 g/mol, 600 g/mol, 650 g/mol, 700 g/mol, 750 g/mol, 800 g/mol, 850 g/mol, 900 g/mol, 950 g/mol, 1000 g/mol, 1250 g/mol, 1500 g/mol, 1750 g/mol, 2000 g/mol, 2500 g/mol, to about 5000 g/mol, or any range derivable therein. In some embodiments, the polyoxyethylene subunit has a molecular weight of about 800 g/mol.

Each of the polyoxyproylene subunits have a molecular weight from about 250 g/mol to about 10000 g/mol, from about 500 g/mol to about 5000 g/mol, from about 750 g/mol to about 3000 g/mol, from about 1500 g/mol to about 2000 g/mol, from about 1600 g/mol to about 1900 g/mol, or from about 1700 g/mol to about 1900 g/mol. The polyoxyproylene subunits may have a subunit from about 250 g/mol, 500 g/mol, 750 g/mol, 1000 g/mol, 1250 g/mol, 1500 g/mol, 1550 g/mol, 1600 g/mol, 1650 g/mol, 1700 g/mol, 1750 g/mol, 1800 g/mol, 1850 g/mol, 1900 g/mol, 1950 g/mol, 2000 g/mol, 2250 g/mol, 2500 g/mol, 2750 g/mol, 3000 g/mol, 3500 g/mol, 4000 g/mol, 4500 g/mol, 5000 g/mol, to about 10000 g/mol, or any range derivable therein. In some embodiments, the polyoxyproylene subunit has a molecular weight of about 1800 g/mol.

In some embodiments, the amount of the pharmaceutically acceptable polymer in the pharmaceutical composition is from about 0.1% w/w to about 25% w/w, from about 0.25% w/w to about 15% w/w, from about 0.4% w/w to about 5% w/w, from about 0.5% w/w to about 2.5% w/w, or from about 1% w/w to about 1.5% w/w. In some embodiments, the amount of the excipient in the pharmaceutical composition is from about 0.1% w/w, 0.2% w/w, 0.25% w/w, 0.3% w/w, 0.4% w/w, 0.5% w/w, 0.6% w/w, 0.7% w/w, 0.75% w/w, 0.8% w/w, 0.9% w/w, 1% w/w, 1.1% w/w, 1.2% w/w, 1.25% w/w, 1.3% w/w, 1.4% w/w, 1.5% w/w, 1.6% w/w, 1.7% w/w, 1.75% w/w, 1.8% w/w, 1.9% w/w, 2% w/w, 2.25% w/w, 2.5% w/w, 5% w/w, 7.5% w/w, 10% w/w, 15% w/w, 20% w/w, to about 25% w/w, or any range derivable therein.

### E. Salts

In some aspects, the present disclosure provides pharmaceutical compositions that contain one or more salts. The salts may be an inorganic potassium or sodium salt such as potassium chloride, sodium chloride, potassium phosphate dibasic, potassium phosphate monobasic, sodium phosphate dibasic, or sodium phosphate monobasic. The pharmaceutical composition may comprise one or more phosphate salts such to generate a phosphate buffer solution. The phosphate buffer solution may be comprise each of the phosphates to buffer a solution to a pH from about 6.8, 6.9, 7.0, 7.1, 7.2, 7.3, 7.4, 7.5, 7.6, 7.7, 7.8, 7.9, or 8.0, or any range derivable therein.

In some embodiments, the amount of the salts in the pharmaceutical composition is from about 0.5% w/w to about 75% w/w, 1% w/w to about 50% w/w, 2% w/w to about 45% w/w, 2.5% w/w to about 40% w/w, 5% w/w to about 30% w/w, 5% w/w to about 10% w/w, or from about 20% w/w to about 30% w/w. In some embodiments, the amount of the slats in the pharmaceutical composition is from about 0.5% w/w, 1% w/w, 2% w/w, 2.5% w/w, 5% w/w, 6% w/w, 7% w/w, 7.5% w/w, 8% w/w, 9% w/w, 10% w/w, 12.5% w/w, 15% w/w, 17.5% w/w, 20% w/w, 22.5% w/w, 25% w/w, 27.5% w/w, 30% w/w, 35% w/w, 40% w/w, 45% w/w, 50% w/w, 60% w/w, 70% w/w, to about 75% w/w, or any range derivable therein.

### F. Excipients

In some aspects, the present disclosure comprises one or more excipients formulated into pharmaceutical compositions. An “excipient” refers to pharmaceutically acceptable carriers that are relatively inert substances used to facilitate administration or delivery of an API into a subject or used to facilitate processing of an API into drug formulations that can be used pharmaceutically for delivery to the site of action in a subject. Furthermore, these compound may be used as diluents in order to obtain a dosage that can be readily measured or administered to a patient. Non-limiting examples of excipients include polymers, proteins, amino acids, stabilizing agents, surfactants, surface modifiers, solubility enhancers, buffers, encapsulating agents, antioxidants, preservatives, nonionic wetting or clarifying agents, viscosity increasing agents, and absorption-enhancing agents. Some specific examples of non-limiting examples of excipients may include Tris buffer, citrate buffer, dextran 40, or a tannin such as a catechin or catechol like epigallocatechin gallate.

In some embodiments, the amount of the additional excipient in the 30 pharmaceutical composition is from about 0.5% w/w to about 95% w/w, or from about 1% w/w to about 85% w/w. In some embodiments, the amount of the excipient in the pharmaceutical composition is from about 0.1% w/w, 0.25% w/w, 0.5% w/w, 1% w/w, 2.5% w/w, 5% w/w, 7.5% w/w, 10% w/w, 15% w/w, 20% w/w, 25% w/w, 30% w/w, 35% w/w, 40% w/w, 45% w/w, 50% w/w, 55% w/w, 60% w/w, 65% w/w, 70% w/w, 75% w/w, 80% w/w, 85% w/w, 90% w/w, to about 95% w/w, or any range derivable therein.

### II. Manufacturing Methods

The final formulations may also be prepared using a thin-film freezing technique. Without wishing to be bound by any theory, it is believed that this process may be used to prepare the dry powder containing all of the elements of composition. The particles obtained from this process may exhibit one or more beneficial properties such as a high surface area, a low tapped density, a low bulk density, or improved flowability or compressibility such as a low Carr's Index. In some aspects, the present disclosure provides methods of preparing a pharmaceutical composition of the present disclosure comprising:
(A) dissolving a mixture of the nucleic acid encapsulated in the lipid nanoparticle, the sugar, and the pharmaceutically acceptable polymer in a solvent to obtain a pharmaceutical mixture;
(B) applying the pharmaceutical mixture to a surface at a surface temperature below 0° C. to obtain a frozen pharmaceutical mixture; and
(C) collecting the frozen pharmaceutical mixture and drying the frozen pharmaceutical mixture to obtain a pharmaceutical composition.

In some embodiments, the pharmaceutical mixture comprises a solid content of the mixture from about 0.05% w/v to about 25% w/v, from about 0.05% w/v to about 5% w/v, from about 0.1% w/v to about 2.5% w/v, 0.15% w/v to about 1.5% w/v, 0.2% w/v to about 0.6% w/v, 0.5% w/v to about 1.25% w/v, or from about 0.050% w/v, 0.075% w/v, 0.10% w/v, 0.125% w/v, 0.150% w/v, 0.175% w/v, 0.200% w/v, 0.225% w/v, 0.250% w/v, 0.275% w/v, 0.300% w/v, 0.325% w/v, 0.350% w/v, 0.375% w/v, 0.400% w/v, 0.425% w/v, 0.450% w/v, 0.475% w/v, 0.500% w/v, 0.525% w/v, 0.550% w/v, 0.575% w/v, 0.600% w/v, 0.650% w/v, 0.700% w/v, 0.750% w/v, 0.800% w/v, 0.900% w/v, 1% w/v, 1.5% w/v, 2% w/v, 4% w/v, 5% w/v, 7.5% w/v, 10% w/v, 12.5% w/v, 15% w/v, 20% w/v, 25% w/v, 30% w/v, 35% w/v, 40% w/v, 45% w/v, to about 50% w/v, or any range derivable therein. In some embodiments, the pharmaceutical mixture is applied at a feed rate from about 0.50 mL/min to about 5.00 mL/min, from about 1.00 mL/min to about 3.00 mL/min, or from about 0.500 mL/min, 0.750 mL/min, 1.00 mL/min, 1.25 mL/min, 1.50 mL/min, 1.75 mL/min, 2.00 mL/min, 2.25 mL/min, 2.50 mL/min, 2.75 mL/min, 3.00 mL/min, 3.25 mL/min, 3.50 mL/min, 3.75 mL/min, 4.00 mL/min, 4.25 mL/min, 4.50 mL/min, 4.75 mL/min, to about 5.00 mL/min, or any range derivable therein.

In some embodiments, the pharmaceutical mixture is applied from a height from about 2 cm to about 50 cm, from about 5 cm to about 20 cm, or from about 1 cm, 2 cm, 4 cm, 6 cm, 8 cm, 10 cm, 12 cm, 14 cm, 16 cm, 18 cm, 20 cm, 21 cm, 22 cm, 23 cm, 24 cm, to about 25 cm, or any range derivable therein. In some embodiments, the surface temperature is from about −190° C. to about 0° C., from about −125° C. to about −25° C., or from about −190° C., −180° C., −170° C., −160° C., −150° C., −140° C., −130° C., −120° C., −110° C., −100° C., −90° C., −80° C., −70° C., −60° C., −50° C., −40° C., −30° C., −20° C., −10° C., to about 0° C., or any range derivable therein. In some embodiments, the surface is rotating at a speed from about 1 rpm to about 1000 rpm, from about 5 rpm to about 500 rpm, from about 100 rpm to about 400 rpm, or from about 5 rpm, 10 rpm, 15 rpm, 25 rpm, 50 rpm, 75 rpm, 100 rpm, 150 rpm, 200 15 rpm, 250 rpm, 300 rpm, 350 rpm, 400 rpm, 450 rpm, to about 500 rpm, or any range derivable therein. In other embodiments, the surface may be stationary.

In some embodiments, the wherein the frozen pharmaceutical composition is dried by lyophilization. In further embodiments, the frozen pharmaceutical composition is dried at a first reduced pressure from about 10 mTorr to 500 mTorr, from about 50 mTorr to about 250 mTorr, or from about 10 mTorr, 20 mTorr, 30 mTorr, 40 mTorr, 50 mTorr, 75 mTorr, 100 mTorr, 150 mTorr, 200 mTorr, 250 mTorr, 300 mTorr, 350 mTorr, 400 mTorr, 450 mTorr, to about 500 mTorr, or any range derivable therein. In some embodiments, the frozen pharmaceutical composition is dried at a first reduced temperature from about −100° C. to about 0° C., from about −60° C. to about −20° C., or from about −100° C., −90° C., −80° C., −70° C., −60° C., −50° C., −40° C., −30° C., −20° C., −10° C., to about 0° C., or any range derivable therein. In some embodiments, the frozen pharmaceutical composition is dried for a primary drying time period from about 3 hours to about 36 hours, from about 6 hours to about 24 hours, or from about 3 hours, 4 hours, 5 hours, 6 hours, 12 hours, 18 hours, 24 hours, 30 hours, to about 36 hours, or any range derivable therein.

In some embodiments, the frozen pharmaceutical composition is dried at a second reduced pressure from about 10 mTorr to 500 mTorr, from about 50 mTorr to about 250 mTorr, or from about 10 mTorr, 20 mTorr, 30 mTorr, 40 mTorr, 50 mTorr, 75 mTorr, 100 mTorr, 150 mTorr, 200 mTorr, 250 mTorr, 300 mTorr, 350 mTorr, 400 mTorr, 450 mTorr, to about 500 mTorr, or any range derivable therein. In some embodiments, the frozen pharmaceutical composition is dried at a second reduced temperature from about 0° C. to about 30° C., from about 10° C. to about 30° C., or from about 0° C., 5° C., 10° C., 15° C., 20° C., 25° C., to about 30° C., or any range derivable therein. In some embodiments, the frozen pharmaceutical composition is dried for a second time for a second time period from about 0.5 hours to about 36 hours, from about 6 hours to about 24 hours, or from about 3 hours, 4 hours, 5 hours, 6 hours, 12 hours, 18 hours, 24 hours, 30 hours, to about 36 hours, or any range derivable therein. In some embodiments, the temperature is changed from the first reduced temperature to the second reduced temperature over a ramping time period from about 3 hours to about 36 hours, from about 6 hours to about 24 hours, or from about 3 hours, 4 hours, 5 hours, 6 hours, 12 hours, 18 hours, 24 hours, 30 hours, to about 36 hours, or any range derivable therein.

### III. Definitions

The use of the word “a” or “an” when used in conjunction with the term “comprising” in the claims and/or the specification may mean “one,” but it is also consistent with the meaning of “one or more,” “at least one,” and “one or more than one.” As used herein “another” may mean at least a second or more.

As used herein, the terms “drug”, “pharmaceutical”, “active agent”, “therapeutic agent”, and “therapeutically active agent” are used interchangeably to represent a compound which invokes a therapeutic or pharmacological effect in a human or animal and is used to treat a disease, disorder, or other condition. In some embodiments, these compounds have undergone and received regulatory approval for administration to a living creature.

The use of the term “or” in the claims is used to mean “and/or” unless explicitly indicated to refer to alternatives only or the alternatives are mutually exclusive. As used herein “another” may mean at least a second or more.

The terms “compositions,” “pharmaceutical compositions,” “formulations,” “pharmaceutical formulations,” “preparations”, and “pharmaceutical preparations” are used synonymously and interchangeably herein.

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

“Subject” and “patient” refer to either a human or non-human, such as primates, mammals, and vertebrates. In particular embodiments, the subject is a human

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “derivative thereof” refers to any chemically modified polysaccharide, wherein at least one of the monomeric saccharide units is modified by substitution of atoms or molecular groups or bonds. In one embodiment, a derivative thereof is a salt thereof. Salts are, for example, salts with suitable mineral acids, such as hydrohalic acids, sulfuric acid or phosphoric acid, for example hydrochlorides, hydrobromides, sulfates, hydrogen sulfates or phosphates, salts with suitable carboxylic acids, such as optionally hydroxylated lower alkanoic acids, for example acetic acid, glycolic acid, propionic acid, lactic acid or pivalic acid, optionally hydroxylated and/or oxo-substituted lower alkanedicarboxylic acids, for example oxalic acid, succinic acid, fumaric acid, maleic acid, tartaric acid, citric acid, pyruvic acid, malic acid, ascorbic acid, and also with aromatic, heteroaromatic or araliphatic carboxylic acids, such as benzoic acid, nicotinic acid or mandelic acid, and salts with suitable aliphatic or aromatic sulfonic acids or N-substituted sulfamic acids, for example methanesulfonates, benzenesulfonates, p-toluenesulfonates or N-cyclohexylsulfamates (cyclamates).

The term “dissolution” as used herein refers to a process by which a solid substance, here the active ingredients, is dispersed and then dissolved in molecular form in a medium. The dissolution rate of the active ingredients of the pharmaceutical dose of the invention is defined by the amount of drug substance that goes in solution per unit time under standardized conditions of liquid/solid interface, temperature and solvent composition.

As used herein, the term “physiological pH” refers to a solution with is at its normal pH in the average human. In most situation, the solution has a pH of approximately 7.4.

As used herein, “dry powder” refers to a fine particulate composition that is not suspended or dissolved in an aqueous liquid.

The term “amorphous” refers to a noncrystalline solid wherein the molecules are not organized in a definite lattice pattern. Alternatively, the term “crystalline” refers to a solid wherein the molecules in the solid have a definite lattice pattern. The crystallinity of the active agent in the composition is measured by powder x-ray diffraction.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

As used in this specification, the term “significant” (and any form of significant such as “significantly”) is not meant to imply statistical differences between two values but only to imply importance or the scope of difference of the parameter.

Throughout this application, the term “about” is used to indicate that a value includes the inherent variation of error for the device, the method being employed to determine the value, or the variation that exists among the study subjects or experimental studies. Unless another definition is applicable, the term “about” refers to ±5% of the indicated value.

As used herein, the term “free of” or “free” in terms of a specified component, is used herein to mean that none of the specified component has been purposefully formulated into a composition and/or is present only as a contaminant or in trace amounts. The total amount of all containments, by-products, and other material is present in that composition in an amount less than 10%. The term “substantially free of” or “substantially free” in terms of a specified component, is used herein to mean that none of the specified component has been purposefully formulated into a composition and/or is present only as a contaminant or in trace amounts. The total amount of all containments, by-products, and other material is present in that composition in an amount less than 5%. The term “essentially free of” or “essentially free” is used to represent that the composition contains less than 2% of the specific component. The term “entirely free of” or “entirely free” contains less than 0.5% of the specific component. Similarly, the term “substantially” means that at least 60% of the composition has the identified property and the term “essentially” means that at least 80% of the composition has the identified property.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements and parameters.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### IV. Examples

To facilitate a better understanding of the present disclosure, the following examples of specific embodiments are given. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. In no way should the following examples be read to limit or define the entire scope of the disclosure.

In addition to the examples shown below, additional examples may include i) evaluation of the integrity and function of the mRNA in the mRNA-LNPs in the dry powders by transfecting cells in culture with the mRNA-LNPs and measuring the expression of the gene(s) encoded by the mRNA, ii) evaluation of the activity of the mRNA-LNPs in the dry powders by administering them to animals or human subjects by needle-based injection after the dry powders are reconstituted with a diluent, and iii) evaluation of the aerosol performance properties of the dry powders and testing the function and activity of the mRNA-LNPs after the dry powders are administered to animals or human subjects by an alternative route of administration such as directly into the nasal cavity, by oral inhalation into the lung using a dry powder inhaler, or insufflation into a cavity such as nasal cavity, eyes, or skin wounds.

### Example 1—Preparation of TFF-mRNA/LNP Dry Powder

Formulation 1: To a scintillation vial, 3.5 mL of poloxamer 188 (1.0 mg/mL) was added, followed by the addition 10.0 mL of an approved mRNA COVID-19 vaccine (reconstituted, 2.567 mg LNP/mL). The mixture was gently shaken and dropped dropwise onto the cryogenically cooled (−180° C.) stainless steel drum. The frozen sample was collected in a stainless-steel container, filled with liquid nitrogen. The sample was transferred in a glass lyophilized vial and stored in a −80° C. freezer until placing in a lyophilizer. The solvent was removed by lyophilizer by a processing of holding at −40° C. for 20 h at or below 100 mTorr, ramping to 25° C. for 20 h at 100 mTorr, and holding at 25° C. for 5 h at 100 mTorr. The dry nitrogen gas was backfilled, and the lid of the vial was closed by the stoppering system before open the lyophilizer door. The vial was sealed with an aluminum cap for storage.

Formulation 2: To a scintillation vial, 10.5 mL of sucrose (20.0 mg/mL) and 4.2 mL of poloxamer 188 (1.0 mg/mL) were added, followed by the addition of 3.0 mL of an approved mRNA COVID-19 vaccine (reconstituted, 2.567 mg LNP/mL). The mixture was gently shaken and dropped dropwise onto the cryogenically cooled (−180° C.) stainless steel drum. The frozen sample was collected in a stainless-steel container, filled with liquid nitrogen. The sample was transferred in a glass lyophilized vial and stored in a −80° C. freezer until placing in a lyophilizer The solvent was removed by lyophilizer by a processing of holding at −40° C. for 20 h at or below 100 mTorr, ramping to 25° C. for 20 h at 100 mTorr, and holding at 25° C. for 5 h at 100 mTorr. The dry nitrogen gas was backfilled, and the lid of the vial was closed by the stoppering system before open the lyophilizer door. The vial was sealed with an aluminum cap for storage.

Formulation 3: To a scintillation vial, 8.0 mL of trehalose (20.0 mg/mL) and 4.6 mL of poloxamer 188 (1.0 mg/mL) were added, followed by the addition of 2.0 mL of an approved mRNA COVID-19 vaccine (reconstituted and dialyzed, 2.127 mg LNP/mL). The mixture was gently shaken and dropped dropwise onto the cryogenically cooled (−180° C.) stainless steel drum. The frozen sample was collected in a stainless-steel container, filled with liquid nitrogen. The sample was transferred in a glass lyophilized vial and stored in a −80° C. freezer until placing in a lyophilizer The solvent was removed by lyophilizer by a processing of holding at −40° C. for 20 h at or below 100 mTorr, ramping to 25° C. for 20 h at 100 mTorr, and holding at 25° C. for 5 h at 100 mTorr. The dry nitrogen gas was backfilled, and the lid of the vial was closed by the stoppering system before open the lyophilizer door. The vial was sealed with an aluminum cap for storage.

Formulation 4: To a scintillation vial, 8.0 mL of sucrose (20.0 mg/mL) and 4.6 mL of poloxamer 188 (1.0 mg/mL) were added, followed by the addition of 2.0 mL of an approved mRNA COVID-19 vaccine (reconstituted and dialyzed, 2.127 mg LNP/mL). The mixture was gently shaken and dropped dropwise onto the cryogenically cooled (−180° C.) stainless steel drum. The frozen sample was collected in a stainless-steel container, filled with liquid nitrogen. The sample was transferred in a glass lyophilized vial and stored in a −80° C. freezer until placing in a lyophilizer The solvent was removed by lyophilizer by a processing of holding at −40° C. for 20 h at or below 100 mTorr, ramping to 25° C. for 20 h at 100 mTorr, and holding at 25° C. for 5 h at 100 mTorr. The dry nitrogen gas was backfilled, and the lid of the vial was closed by the stoppering system before open the lyophilizer door. The vial was sealed with an aluminum cap for storage.

Formulation 5: To a 200 μL centrifuge tube, 40 μL of sucrose (20.0 mg/mL) and 13 μL of poloxamer 188 (1.0 mg/mL) were added, followed by the addition of 10 μL of an approved mRNA COVID-19 vaccine (reconstituted and dialyzed, 2.16 mg LNP/mL). The mixture was gently shaken and dropped dropwise onto the cryogenically cooled (−180° C.) stainless steel drum. The frozen sample was collected in a stainless-steel container, filled with liquid nitrogen. The sample was transferred in a glass lyophilized vial and stored in a −80° C. freezer until placing in a lyophilizer The solvent was removed by lyophilizer by a processing of holding at −40° C. for 20 h at or below 100 mTorr, ramping to 25° C. for 20 h at 100 mTorr, and holding at 25° C. for 5 h at 100 mTorr. The dry nitrogen gas was backfilled, and the lid of the vial was closed by the stoppering system before open the lyophilizer door. The vial was sealed with an aluminum cap for storage.

Shelf freeze-drying: For mRNA-LNP formulations 1 and 2 as mentioned above, dry powders were also prepared with conventional shelf freeze-drying. The mRNA-LNPs in suspension (0.6 mL) were placed into 2 mL lyophilized vials and the vials were placed in a Advantage EL shelf freeze dryer. The shelf temperature was cooled from room temperature to −50° C. at the rate of 1° C./min, and maintained at 50° C. for lh before drying. The drying cycle was the same as one used to sublime water from the thin-film frozen samples.

### Example 2—Dialysis

The approved mRNA COVID vaccines were dialyzed against at least 1,000 fold-volume of diethyl pyrocarbonate (DEPC)-treated water at 4° C. for 24 h. The concentration of LNPs was then adjusted based on the volume change after dialysis.

For example, 1.200 mL of the approved mRNA COVID vaccine was placed into a dialysis tube (Spectrum, Stamford, CT), then the dialysis tube was placed in 1,500 mL of DEPC-treated water in an external beaker with a gentle stirring speed of 100 rpm at 4° C. for 24 h. The dialysis solution (i.e. DEPC-treated water) was changed every 8 h. Finally, 1.398 mL of sample was recovered from the dialysis tube. The concentration of LNPs was calculated based on the volume change for the formulation preparation for TFF.

### Example 3—Characterization of the TFF-mRNA/LNP Dry Powder

### A. Particle Size Distribution (PSD)

A small quantity of TFF powder was placed into a disposable UV cuvette and reconstituted with filtered water (Evoqua, Warrendale, PA). Particle size distribution was measured using a Zetasizer Nano ZS (Malvern Panalytical Ltd, Malvern, UK) with dispersant refractive index of 1.33 and material refractive index of 1.45. Shown in Table 1 below are the particle size (Z-average) of the mRNA-LNPs before they were subjected to thin-film freeze-drying (TFFD), after they were subjected to TFFD and reconstitution, and after the dry powders were storated at in a refrigerator (˜4° C.) or at temperature (˜25° C.) for three weeks.

TABLE 1
Particle size distribution of dry powder. Data are mean ± SD (n = 3).

| | | Z-average (d · nm) | | | |
|---|---|---|---|---|---|
| | Freezing | | 25° C., | 4° C., | 4° C., |
| Formulation | Method | Initial | 3 weeks | 3 weeks | 5 weeks |
| mRNA/LNP | — | 87.8 ± 2.2 | — | — | — |
| COVID Vac | | | | | |
| | Shelf-freeze | 235 | — | — | — |
| mRNA/LNP | — | 95.2 ± 0.4 | — | — | — |
| COVID Vac, | | | | | |
| Dialyzed | | | | | |
| Formulation 1 | TFF | 122.9 ± 3.5 | 144.0 ± 2.2 | 117.9 ± 1.7 | 130.2 ± 1.37 |
| | Shelf-freeze | 225 | — | — | — |
| Formulation 2 | TFF | 151.7 ± 3.6 | 172.7 ± 3.3 | 164.1 ± 5.8 | 151.2 ± 0.49 |
| | Shelf-freeze | 123 | — | — | — |
| Formulation 3 | TFF | 112.0 ± 1.1 | 144.4 ± 1.9 | 115.4 ± 0.9 | — |
| Formulation 4 | TFF | 116.9 ± 2.0 | 244.3 ± 3.3 | 125.5 ± 1.3 | — |
| Formulation 5 | TFF | 118.8 | — | — | — |

B. Quantification of mRNA Encapsulation Efficiency

The mRNA loading in an approved mRNA/LNP COVID vaccine formulations was quantified using a Quanti-iT RiboGreen assay kit (Invitrogen, Carlsbad, CA) as previously described (Blakney et al., 2019; Yang et al., 2020). Powder samples were reconstituted to the same concentration as the liquid formulations before TFF process. All samples were diluted two, twenty, two-hundred, and two-thousand times in 1×TE buffer (RNase-free) containing 0.5% (v/v) Triton X-100 (Sigma Aldrich, St. Louis, MO) for a 15 min of incubation to detect total mRNA. For detecting free mRNA, all samples were diluted two, twenty, two-hundred, and two-thousand time in lx TE buffer (RNase-free). All the Triton X-100 treated and untreated samples were incubated with RiboGreen reagent in a black, 96 well-plate (Costar, Corning, NY). The fluorescence intensity was recorded by a BioTek Synergy HT Multi-Mode Microplate Reader (Winooski, VT, Ex=485 nm, Em=528 nm, gain=35). Fluorscence intensity values were converted to mRNA concentrations based on standard curves built for total mRNA and mRNA outside of LNPs, respectively. The encapsulation efficiency was calculated according to the following formula:

$$Encapsulation efficiency ( E E , \% ) = \frac{total m R N A - free m R N A}{total m R N A} \times 100 \%$$

TABLE 2
Encapsulation efficiency

| | | Encapsulation |
|---|---|---|
| Formulation | | (%) |
| mRNA/LNP COVID Vac | Reconstituted, original Vac | 87.9 |
| Formulation 1 | Before TFF | 91.5 |
| | After TFF, dried and | 93.0 |
| | reconstituted | |

### C. Transmission Electron Microscope (TEM) Analysis

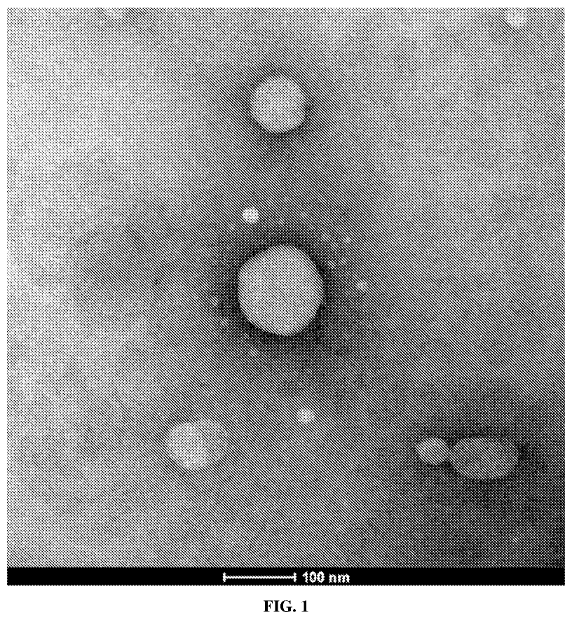

The morphology of LNP formulations was studied using FEI Tecnai transmission electron microscopy. Thin-film freeze-dried mRNA/LNP powder was reconstituted in water and diluted with purified water to obtain a LNP concentration of 0.1-0.3 mg/mL. Five μL of LNP dispersion was added on a 200-mesh carbon film, copper grid (Electron Microscopy Sciences, Hatfield, PA). After one minute, a filter paper was used to gently remove the liquid from the edge of the grid. Five μL of 1% phosphotungstic acid was dropped on the grid to negatively stain the sample. After one minute, a filter paper was used to remove the stain from the edge of the grid. The sample was air-dried before capturing images. See FIG. 1.

### Example 4—Poly(A)-Lipid Nanoparticle Preparation, Characterization, and Thin-Film Freeze-Drying

A. Preparation of mRNA-Lipid Nanoparticles

Lipid nanoparticles (LNPs) loaded with mRNA were prepared by combining an aqueous solution of poly(A) (Roche Diagnostics, Mannheim, Germany) in a 25 mM sodium citrate buffer (pH 3) with an ethanolic solution of lipids containing the ionizable lipid DLin-MC3-DMA (MedChemExpress, Monmouth Junction, NJ), DSPC (Avanti Polar lipids, Alabaster, AL), cholesterol (Sigma Aldrich, St. Louis, MO), and PEG₂₀₀₀-DSPE (Avanti Polar lipids) at molar ratio of 40:10:45:5, respectively. A microfluidics flow control system (i.e., PG-MFC controller, PreciGenome, San Jose, CA) was used to combine the aqueous and organic phases. The concentration of poly(A) in the aqueous phase was 0.189 mg/mL, while the total lipid concentration in the organic phase was 7.56 mg/mL (i.e., 12.5 mM). The ratio of poly(A) to total lipids was 1:20 w/w. The total flow rate was 12 mL/min and the flow rate ratio (aqueous: organic) was 2:1 v/v. The final poly(A) concentration in the aqueous/ethanolic phase that contains the synthesized LNPs was 63 μg/mL.

### B. Dialysis of Poly(A)-LNPs

One milliliter (1 mL) of poly(A)-LNPs in the aqueous/ethanol phase were placed in a dialysis tube with a cut-off of molecular weight of 8-10 KDa (Spectrum, Stamford, CT). The dialysis tube was then placed in 1000 mL of DEPC-treated 1×PBS (i.e., 10 mM, pH 7.4) in an external beaker with a gentle stirring speed of 100 rpm at 4° C. for 24 h. The dialysis solution (i.e., DEPC-treated 1×PBS) was changed every 8 h. After dialysis, 1.602 mL of sample was recovered from the dialysis tube. A sucrose stock solution was added to reach a final concentration of 10% (w/v) as cryoprotectant. The poly(A)-LNPs samples were aliquoted Eppendorf tube (0.2 mL each) and then frozen and stored in a −80° C. freezer. The concentration of LNPs was calculated based on the volume change after the dialysis and addition of sucrose for the formulation preparation for TFF. Meanwhile, the poly(A)-LNPs in original buffer (i.e. a mixture of sodium citrate buffer and ethanol) without dialysis was also formulated for TFF.

### C. Preparation of Thin-Film Freeze-Dried Poly(A)-LNP Powders

Formulation 6: To a 1.5 mL of Eppendorf tube, 0.05 mL of poloxamer 188 (60 mg/mL), 0.05 mL of trehalose (400 mg/mL) and 0.05 mL of leucine (20 mg/mL) were added and pipetted thoroughly, followed by the addition 0.1 mL of poly(A)-LNPs in sodium citrate buffer/ethanol mixture with a concentration of 0.063 mg poly(A)/mL.

Formulation 7: To a 1.5 mL of Eppendorf tube, 0.05 mL of poloxamer 188 (60 mg/mL), 0.05 mL of sucrose (400 mg/mL) and 0.05 mL of leucine (20 mg/mL) were added and pipetted thoroughly, followed by the addition 0.1 mL of poly(A)-LNPs in sodium citrate buffer/ethanol mixture with a concentration of 0.063 mg poly(A)/mL.

Formulation 8: To a 1.5 mL of Eppendorf tube, 0.05 mL of poloxamer 188 (60 mg/mL), 0.025 mL of sucrose (400 mg/mL), 0.025 mL of trehalose (400 mg/mL), and 0.05 mL of leucine (20 mg/mL) were added and pipetted thoroughly, followed by the addition 0.1 mL of poly(A)-LNPs in sodium citrate buffer/ethanol mixture with a concentration of 0.063 mg poly(A)/mL.

Formulation 9: To a 1.5 mL of Eppendorf tube, 0.05 mL of poloxamer 188 (60 mg/mL), 0.05 mL of trehalose (400 mg/mL), and 0.05 mL of leucine (20 mg/mL) were added and pipetted thoroughly, followed by the addition 0.1 mL of dialyzed poly(A)-LNPs with a concentration of 0.039 mg poly(A)/mL.

Formulation 10: To a 1.5 mL of Eppendorf tube, 0.05 mL of poloxamer 188 (60 mg/mL), 0.05 mL of sucrose (400 mg/mL), and 0.05 mL of leucine (20 mg/mL) were added and pipetted thoroughly, followed by the addition 0.1 mL of dialyzed poly(A)-LNPs with a concentration of 0.039 mg poly(A)/mL.

Formulation 11: To a 1.5 mL of Eppendorf tube, 0.05 mL of poloxamer 188 (60 mg/mL), 0.025 mL of sucrose (400 mg/mL), 0.025 mL of trehalose (400 mg/mL), and 0.05 mL of leucine (20 mg/mL) were added and pipetted thoroughly, followed by the addition 0.1 mL of dialyzed poly(A)-LNPs with a concentration of 0.039 mg poly(A)/mL.

### D. Thin-Film Freezing and Sublimation

The poly(A)-LNP dispersions were gently swirled for a few seconds and

dropped dropwise (0.250 mL in 9-10 drops) onto the cryogenically cooled stainless-steel drum (−70° C.). The frozen films were collected in a stainless-steel container filled with liquid nitrogen. The frozen films were then transferred into a glass vial and stored in a −80° C. freezer before being placed into a VirTis Advantage bench top tray lyophilizer (The VirTis Company, Inc. Gardiner, NY). The lyophilization cycle was −40° C. for 20 h at 80 mTorr, ramping to 25° C. in 20 h at 80 mTorr, and then holding at 25° C. for 20 h at 80 mTorr. Dry nitrogen gas was backfilled, and the lid of the vial was closed by the stoppering system. The vial was sealed with an aluminum cap for storage.

E. Characterization of Poly(A)-LNPs Reconstituted from TFFD Powders

TFFD powder was placed into a disposable UV cuvette and reconstituted

with filtered water deionized water. Particle size and polydispersity index (PDI) were measured using a Malvern Zetasizer Nano ZS. The mRNA loading in the poly(A)-LNPs was quantified using a Quanti-iT RiboGreen assay kit (Invitrogen, Carlsbad, CA) as previously described (Blakney et al., 2019; Yang et al., 2020). Briefly, TFFD powder was reconstituted with a citrate buffer (50 mM, pH 4.0) and then diluted with 1×PBS (10 mM, pH 7.4) to a volume twice of the liquid formulations before TFFD process. All samples were diluted 2-, 20-, 200-, and 2000- fold in 1×Tris-EDTA (TE) buffer (RNase-free) containing 0.5% (v/v) Triton X-100 (Sigma Aldrich, St. Louis, MO) and then incubated for 15 min to detect total mRNA. To detect free, encapsulated mRNA, all samples were diluted 2-, 20-, 200-, and 2000-fold in 1×TE buffer (RNase-free). All the Triton X-100 treated and untreated samples were incubated with the RiboGreen reagent in a black, 96 well-plate (Costar, Corning, NY). The fluorescence intensity was measured using a BioTek Synergy HT Multi-Mode Microplate Reader (Winooski, VT, Ex=485 nm, Em=528 nm, gain=35). Fluorescence intensity values were converted to mRNA concentrations based on standard curves built for total mRNA and mRNA outside of LNPs, respectively. The encapsulation efficiency was calculated according to the following formula:

$$Encapsulation efficiency ( E E , \% ) = \frac{total m R N A - free m R N A}{total m R N A} \times 100 \%$$

i. Results

Shown in Table 1 are the particle size (Z-average), PDI, and encapsulation efficacy of the poly(A)-LNPs after dialysis into a sucrose in 1×PBS with 10% (w/v) solution and after they were reconstituted from formulations 6-11 as mentioned above. Overall, the encapsulation efficacy of the poly(A) in the LNPs after being subjected to TFFD and reconstitution did not change (as compared to the value after dialysis). The particle size of the poly(A)-LNPs in formulations 9-11 also did not change (Table 3). However, the particle size of the poly(A)-LNPs in formulations 6-8, which was prepared with poly(A)-LNPs dispersed in a citrate buffer and ethanol solution, increased significantly.

TABLE 3
Particle size, PDI, and encapsulation efficacy of poly(A)-
LNPs after they were subjected to TFFD. Data are
mean ± SD of three measurements for Z-average and
PDI and two measurements for encapsulation efficacy.

| | Z-average | | Encapsulation |
|---|---|---|---|
| Formulation | (d · nm) | PDI | efficacy (%) |
| Poly(A)-LNPs after | 143.2 ± 6.2 | 0.3 ± 0.0 | 68.6 ± 0.1 |
| dialysis | | | |
| Formulation 6 | 499.1 ± 254.2 | 0.6 ± 0.1 | 67.3 ± 1.2 |
| Formulation 7 | 279.6 ± 13.3 | 0.4 ± 0.1 | 72.0 ± 2.3 |
| Formulation 8 | 263.2 ± 23.2 | 0.4 ± 0.0 | 66.6 ± 0.5 |
| Formulation 9 | 152.7 ± 14.5 | 0.3 ± 0.1 | 66.6 ± 1.1 |
| Formulation 10 | 144.0 ± 20.5 | 0.3 ± 0.0 | 67.9 ± 0.1 |
| Formulation 11 | 142.1 ± 18.5 | 0.3 ± 0.1 | 73.2 ± 0.8 |

### Example 5—Thin-Film Freeze-Drying of mRNA-LNPs Re-Dialyzed to Remove PBS and Sucrose

### A. Dialysis of Poly(A)-LNPs and Preparation of Thin-Film Freeze-Dried Poly(A)-LNP Powders

Poly(A)-LNPs in a sucrose (10%, w/v) in PBS (10 mM, pH 7.4) were stored in −80° C. for two months. They were then dialyzed in DEPC-treated water as the dialysis solution for 16 hours, and the dialysis solution was changed every 8 h. The concentration of the poly(A)-LNPs was calculated based on the volume change after the dialysis for the formulation preparation for TFF. Stock solutions of 1000 mg/mL of sucrose, 500 mg/mL of trehalose, 20 mg/mL of leucine, 200 mg/mL of mannitol, 1500 mg/mL of sorbitol, 1200 mg/mL of xylitol, 80 mg/mL of P188, 1M of Tris buffer, 10×PBS, 20 mg/mL of dextran-40, and 4 mg/mL of epigallocatechin gallate (EGCG) were prepared and used to prepare poly(A)-LNP formulation (Table 4) for TFFD.

TABLE 2
Formulation of poly(A)-LNPs (* volume of poly(A)-LNPs re-dialyzed in water).

| | Volume | | | | | | | | | | | | |
|---|---|---|---|---|---|---|---|---|---|---|---|---|---|
| | of | | | | | | | | | | | | |
| | poly(A)- | Volume of stock solution added (μL) | | | | | | | | | | | |
| | LNPs | | | | | | | | Tris | | Dextran- | | |
| Formulation | (μL)* | Sucrose | Trehalose | Leucine | Mannitol | Sorbitol | Xylitol | P188 | buffer | PBS | 40 | EGCG | diH2O |
| Poly(A)- | 10 | | | | | | | | | | | | 190 |
| LNPs after | | | | | | | | | | | | | |
| dialysis in | | | | | | | | | | | | | |
| DEPC-water | | | | | | | | | | | | | |
| Formulation | 10 | 10 | 20 | 50 | | | | 37.5 | | | | | 72.5 |
| 12 | | | | | | | | | | | | | |
| Formulation | 10 | 30 | 40 | 50 | | | | 37.5 | | | | | 32.5 |
| 13 | | | | | | | | | | | | | |
| Formulation | 10 | 50 | | 50 | | | | 37.5 | | | | | 52.5 |
| 14 | | | | | | | | | | | | | |
| Formulation | 10 | 80 | | 50 | | | | 37.5 | | | | | 22.5 |
| 15 | | | | | | | | | | | | | |
| Formulation | 10 | 100 | | 50 | | | | 37.5 | | | | | 2.5 |
| 16 | | | | | | | | | | | | | |
| Formulation | 10 | | 80 | 50 | | | | 37.5 | | | | | 22.5 |
| 17 | | | | | | | | | | | | | |
| Formulation | 10 | | 40 | 50 | | | | 37.5 | | | | | 62.5 |
| 18 | | | | | | | | | | | | | |
| Formulation | 10 | | | 50 | 75 | | | 37.5 | | | | | 27.5 |
| 19 | | | | | | | | | | | | | |
| Formulation | 10 | | | 50 | | 40 | | 37.5 | | | | | 62.5 |
| 20 | | | | | | | | | | | | | |
| Formulation | 10 | | | 50 | | 80 | | 37.5 | | | | | 22.5 |
| 21 | | | | | | | | | | | | | |
| Formulation | 10 | | | 50 | | | 100 | 37.5 | | | | | 2.5 |
| 22 | | | | | | | | | | | | | |
| Formulation | 10 | 30 | | 50 | 75 | | | 37.5 | | | | | 0 |
| 23 | | | | | | | | | | | | | |
| Formulation | 10 | 30 | 40 | 50 | | | | 37.5 | | | 30 | | 2.5 |
| 24 | | | | | | | | | | | | | |
| Formulation | 10 | 30 | 40 | 50 | | | | 37.5 | 4 | | | | 28.5 |
| 25 | | | | | | | | | | | | | |
| Formulation | 10 | 30 | 40 | 50 | | | | 37.5 | 1 | | | | 31.5 |
| 26 | | | | | | | | | | | | | |
| Formulation | 10 | 30 | 40 | 50 | | | | 37.5 | | 20 | | | 12.5 |
| 27 | | | | | | | | | | | | | |
| Formulation | 10 | 30 | 40 | 50 | | | | 37.5 | | | | 30 | 2.5 |
| 28 | | | | | | | | | | | | | |

### C. Thin-Film Freezing and Sublimation

The poly(A)-LNP dispersions in Table 4 were dropped dropwise onto the cryogenically cooled stainless-steel drum (−70 ° C.). The frozen films were collected in a stainless-steel container filled with liquid nitrogen. The frozen films were then transferred into a glass vial and stored in a −80° C. freezer before placing into a VirTis Advantage bench top tray lyophilizer. Traditional lyophilization procedures were utilized. Dry nitrogen gas was backfilled, and the lid of the vial was closed by the stoppering system. The vial was sealed with an aluminum cap for storage.

D. Characterization of Poly(A)-LNPs Reconstituted from TFFD Powders

TFFD powders were reconstituted with deionized water to the same concentration as the liquid formulations before TFFD process. The particle size and PDI were measured as previously described using a Malvern Nano ZS.

i. Results

The particle size and PDI values of the poly(A)-LNPs in formulations 12-28 upon reconstitution with water are shown in Table 5. The additional dialysis step to remove the PBS and sucrose from the poly(A)-LNP dispersion led to an increase in the particle size of the LNPs (Table 5 vs. Table 3). For the further dialyzed poly(A)-LNPs, the excipients added into the dispersion affected the particle size of the poly(A)-LNPs after they were subjected to TEND and then reconstitution in water (Table 5). For example, the particle sizes of the poly(A)-LNPs in formulations 14, 16, and 17 did not increase after they were subjected to TEND and reconstitution, while they became much larger in formulations 25 and 26 (Table 5).

TABLE 5
Particle size and PDI of poly(A)-LNPs after they were subjected
to TFFD. Data are mean ± SD from two measurements.

| Formulation | Z-average (d · nm) | PDI |
|---|---|---|
| Poly(A)-LNPs after dialysis | 246.9 ± 6.4 | 0.4 ± 0.0 |
| in DEPC-water | | |
| Formulation 12 | 914.8 ± 394.8 | 0.7 ± 0.3 |
| Formulation 13 | 638.9 ± 87.0 | 0.6 ± 0.1 |
| Formulation 14 | 253.5 ± 25.8 | 0.3 ± 0.0 |
| Formulation 15 | 323.4 ± 9.6 | 0.4 ± 0.0 |
| Formulation 16 | 257.7 ± 28.9 | 0.4 ± 0.1 |
| Formulation 17 | 227.7 ± 6.3 | 0.3 ± 0.0 |
| Formulation 18 | 409.5 ± 12.2 | 0.5 ± 0.1 |
| Formulation 19 | 335.3 ± 83.8 | 0.4 ± 0.1 |
| Formulation 20 | 451.7 ± 237.5 | 0.5 ± 0.2 |
| Formulation 21 | 503.9 ± 228.7 | 0.5 ± 0.2 |
| Formulation 22 | 562.5 ± 362.1 | 0.6 ± 0.2 |
| Formulation 23 | 289.6 ± 8.3 | 0.4 ± 0.0 |
| Formulation 24 | 434.5 ± 175.0 | 0.4 ± 0.2 |
| Formulation 25 | 1900.5 ± 9.2 | 1.0 ± 0.0 |
| Formulation 26 | 1503.0 ± 261.6 | 0.9 ± 0.1 |
| Formulation 27 | 776.2 ± 65.0 | 0.7 ± 0.0 |
| Formulation 28 | 622.1 ± 287.0 | 0.6 ± 0.3 |

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
Blakney et al., Gene Therapy, 26(9):363-372, 2019.
Yang et al., Bioactive Materials, 5(4):1053-1061, 2020.

What is claimed is:

1. A pharmaceutical composition comprising:
(A) a nucleic acid;
(B) one or more lipids sufficient to form a lipid nanoparticle;
(C) a sugar; and
(D) a pharmaceutically acceptable polymer;
wherein the pharmaceutical composition is formulated as a powder and wherein the nucleic acid is substantially encapsulated in the lipid nanoparticle.

2. The pharmaceutical composition of claim 1, wherein the powder is a dry powder.

3. The pharmaceutical composition of claim 1, wherein the powder is free of any water.

4. The pharmaceutical composition of claim 3, wherein the powder is substantially free of any water.

5. The pharmaceutical composition of claim 4, wherein the powder is essentially free of any water.

6. The pharmaceutical composition according to any one of claim 1-5, wherein the nucleic acid is an mRNA.

7. The pharmaceutical composition of claim 6, wherein the nucleic acid is an mRNA that encodes for an antigen.

8. The pharmaceutical composition of claim 7, wherein the antigen is an anti-viral antigen.

9. The pharmaceutical composition of claim 8, wherein the anti-viral antigen is a SARS CoV2 antigen.

10. The pharmaceutical composition of claim 9, wherein the SARS CoV2 antigen is an mRNA which encodes for the SARS CoV2 spike protein or a modified version thereof.

11. The pharmaceutical composition of claim 10, wherein the mRNA encodes for a modified version of the SARS CoV2 spike protein.

12. The pharmaceutical composition of claim 11, wherein the modified version of the SARS CoV2 spike protein contains one or more proline substitutions.

13. The pharmaceutical composition according to any one of claims 1-12, wherein the nucleic acid contains one or more modifications.

14. The pharmaceutical composition of claim 13, wherein the nucleic acid comprises one, two, three, four, five, six, seven, eight, nine, or ten modifications.

15. The pharmaceutical composition of either claim 13 or claim 14, wherein the modifications comprise a 5′ cap, one or more untranslated regions, a signal peptide, a linker sequence, a poly(A) tail, a modified nucleotide, or a series of the same nucleotides.

16. The pharmaceutical composition of claim 15, wherein the modifications include a 5′ untranslated region or a 3′ untranslated region.

17. The pharmaceutical composition according to any one of claims 1-16, wherein the pharmaceutical composition comprises one, two, three, four, five, six, seven, or eight lipids.

18. The pharmaceutical composition of claim 17, wherein the pharmaceutical composition comprises three, four, or five lipids.

19. The pharmaceutical composition according to any one of claims 1-18, wherein the pharmaceutical composition comprises a first lipid.

20. The pharmaceutical composition of claim 19, wherein the first lipid is a lipid with two or more hydrophobic groups.

21. The pharmaceutical composition according to any one of claims 1-20, wherein the pharmaceutical composition comprises a second lipid.

22. The pharmaceutical composition of claim 21, wherein the second lipid is a phospholipid.

23. The pharmaceutical composition of claim 22, wherein the phospholipid is phosphocholine.

24. The pharmaceutical composition of claim 23, wherein the phospholipid is distearoylphosphatidylcholine (DSPC).

25. The pharmaceutical composition according to any one of claims 1-24, wherein the pharmaceutical composition comprises a third lipid.

26. The pharmaceutical composition of claim 25, wherein the third lipid is a PEGylated lipid.

27. The pharmaceutical composition of claim 26, wherein the PEGylated lipid comprises one or more polyethylene glycol units.

28. The pharmaceutical composition of claim 27, wherein the PEGylated lipid comprises a polyethylene glycol unit with a molecular weight from about 1 kilodalton to about 10 kilodaltons.

29. The pharmaceutical composition of claim 28, wherein the PEGylated lipid is N,N-dimyristylamide of 2-hydroxyacetic acid with a polyethylene glycol unit with a molecular weight of about 2 kilodaltons.

30. The pharmaceutical composition of claim 29, wherein the PEGylated lipid is N,N-ditetradecylacetamide with a polyethylene glycol unit with a molecular weight of about 2 kilodaltons.

31. The pharmaceutical composition according to any one of claims 1-30, wherein the pharmaceutical composition comprises a fourth lipid.

32. The pharmaceutical composition of claim 31, wherein the fourth lipid is a steroid.

33. The pharmaceutical composition of claim 32, wherein the steroid is a sterol.

34. The pharmaceutical composition of claim 33, wherein the sterol is cholesterol.

35. The pharmaceutical composition according to any one of claims 1-34, wherein the first, second, third, and fourth lipids form a lipid nanoparticle.

36. The pharmaceutical composition according to any one of claims 1-35, wherein the nucleic acid is essentially encapsulated in the lipid nanoparticle.

37. The pharmaceutical composition according to any one of claims 1-35, wherein the nucleic acid is entirely encapsulated in the lipid nanoparticle.

38. The pharmaceutical composition according to any one of claims 1-37, wherein the sugar is a polysaccharide.

39. The pharmaceutical composition of claim 38, wherein the polysaccharide is a disaccharide or a trisaccharide.

40. The pharmaceutical composition of either claim 38 or claim 39, wherein the polysaccharide is a disaccharide.

41. The pharmaceutical composition of claim 40, wherein the disaccharide contains a glucose.

42. The pharmaceutical composition of either claim 40 or claim 41, wherein the disaccharide is sucrose or trehalose.

43. The pharmaceutical composition of claim 42, wherein the disaccharide is sucrose.

44. The pharmaceutical composition according to any one of claims 1-37, wherein the sugar is a sugar alcohol.

45. The pharmaceutical composition of claim 44, wherein the sugar is mannitol, sorbitol, and xylitol.

46. The pharmaceutical composition according to any one of claims 1-45, wherein the pharmaceutically acceptable polymer is a copolymer.

47. The pharmaceutical composition of claim 46, wherein the copolymer is a triblock copolymer.

48. The pharmaceutical composition of either claim 46 or claim 47, wherein the copolymer comprises one or more polyoxypropylene units and one or more polyoxyethylene units.

49. The pharmaceutical composition according to any one of claims 46-48, wherein the copolymer comprises one polyoxypropylene unit and two polyoxyethylene units.

50. The pharmaceutical composition according to any one of claims 46-49, wherein the copolymer comprises a polyoxypropylene unit with a polyoxyethylene unit on each side of the polyoxypropylene unit.

51. The pharmaceutical composition according to any one of claims 46-50, wherein the polyoxypropylene unit has a molecular weight from about 500 g/mol to about 5000 g/mol.

52. The pharmaceutical composition of claim 51, wherein the molecular weight of the polyoxypropylene unit is from about 750 g/mol to about 3000 g/mol.

53. The pharmaceutical composition of claim 52, wherein the molecular weight of the polyoxypropylene unit is from about 1500 g/mol to about 2000 g/mol.

54. The pharmaceutical composition of claim 53, wherein the molecular weight of the polyoxypropylene unit is about 1800 g/mol.

55. The pharmaceutical composition according to any one of claims 46-54, wherein each of the polyoxyethylene unit has a molecular weight from about 100 g/mol to about 2500 g/mol.

56. The pharmaceutical composition of claim 55, wherein the molecular weight of the polyoxyethylene unit is from about 250 g/mol to about 2000 g/mol.

57. The pharmaceutical composition of claim 56, wherein the molecular weight of the polyoxyethylene unit is from about 600 g/mol to about 1000 g/mol.

58. The pharmaceutical composition of claim 57, wherein the molecular weight of the polyoxyethylene unit is about 800 g/mol.

59. The pharmaceutical composition according to any one of claims 1-58, wherein the pharmaceutically acceptable polymer is poloxamer P188.

60. The pharmaceutical composition according to any one of claims 1-59, wherein the pharmaceutical composition further comprises one or more salts.

61. The pharmaceutical composition of claim 60, wherein the salt is a phosphate buffer.

62. The pharmaceutical composition of either claim 60 or claim 61, wherein the salt is sodium chloride.

63. The pharmaceutical composition according to any one of claims 60-62, wherein the salt is the solids content from phosphate buffered saline (PBS).

64. The pharmaceutical composition according to any one of claims 1-63, wherein the pharmaceutical composition comprises from about 0.1% w/w to about 50% w/w of the lipid nanoparticles.

65. The pharmaceutical composition of claim 64 comprising from about 1% w/w to about 15% w/w of the lipid nanoparticles.

66. The pharmaceutical composition of claim 65 comprising from about 2% w/w to about 10% w/w of the lipid nanoparticles.

67. The pharmaceutical composition of claim 66 comprising from about 2% w/w to about 5% w/w of the lipid nanoparticles.

68. The pharmaceutical composition of claim 66 comprising from about 6% w/w to about 10% w/w of the lipid nanoparticles.

69. The pharmaceutical composition according to any one of claims 1-68, wherein the pharmaceutical composition comprises from about 25% w/w to about 98% w/w of the sugar.

70. The pharmaceutical composition of claim 69 comprising from about 40% w/w to about 95% w/w of the sugar.

71. The pharmaceutical composition of claim 70 comprising from about 50% w/w to about 90% w/w of the sugar.

72. The pharmaceutical composition of claim 71 comprising from about 50% w/w to about 70% w/w of the sugar.

73. The pharmaceutical composition of claim 71 comprising from about 80% w/w to about 90% w/w of the sugar.

74. The pharmaceutical composition according to any one of claims 1-73, wherein the pharmaceutical composition comprises from about 0.1% w/w to about 25% w/w of the pharmaceutically acceptable polymer.

75. The pharmaceutical composition of claim 74 comprising from about 0.25% w/w to about 15% w/w of the pharmaceutically acceptable polymer.

76. The pharmaceutical composition of claim 75 comprising from about 0.4% w/w to about 5% w/w of the pharmaceutically acceptable polymer.

77. The pharmaceutical composition of claim 76 comprising from about 0.5% w/w to about 2.5% w/w of the pharmaceutically acceptable polymer.

78. The pharmaceutical composition of claim 77 comprising from about 1.0% w/w to about 1.5% w/w of the pharmaceutically acceptable polymer.

79. The pharmaceutical composition according to any one of claims 1-78, wherein the pharmaceutical composition comprises from about 1% w/w to about 50% w/w of each salt.

80. The pharmaceutical composition of claim 79 comprising from about 2% w/w to about 45% w/w of each salt.

81. The pharmaceutical composition of claim 80 comprising from about 2.5% w/w to about 40% w/w of each salt.

82. The pharmaceutical composition of claim 81 comprising from about 5% w/w to about 30% w/w of each salt.

83. The pharmaceutical composition of claim 82 comprising from about 5% w/w to about 10% w/w of each salt.

84. The pharmaceutical composition of claim 82 comprising from about 20% w/w to about 30% w/w of each salt.

85. The pharmaceutical composition according to any one of claims 1-84, wherein the pharmaceutical composition comprises one or more particles.

86. The pharmaceutical composition of claim 85, wherein each of the particles comprise the lipid nanoparticles, the pharmaceutically acceptable polymer, and the sugar.

87. The pharmaceutical composition according to any one of claims 1-86, wherein the mRNA recovery after processing is greater than 60%.

88. The pharmaceutical composition of claim 87, wherein the mRNA recovery is greater than 70%.

89. The pharmaceutical composition of claim 88, wherein the mRNA recovery is greater than 80%.

90. The pharmaceutical composition according to any one of claims 1-89, wherein the lipid nanoparticles have a Z-average is from about 50 nm to about 250 nm.

91. The pharmaceutical composition of claim 90, wherein the Z-average is from about 75 nm to about 200 nm.

92. The pharmaceutical composition of claim 91, wherein the Z-average is from about 80 nm to about 150 nm.

93. The pharmaceutical composition according to any one of claims 1-92, wherein the pharmaceutical composition shows less than 10% degradation after 1 month when stored at a temperature below 30° C.

94. The pharmaceutical composition of claim 93, wherein the pharmaceutical composition showed less than 5% degradation after 1 month.

95. The pharmaceutical composition of claim 94, wherein the pharmaceutical composition showed less than 3% degradation after 1 month.

96. The pharmaceutical composition of claim 95, wherein the pharmaceutical composition showed less than 1% degradation after 1 month.

97. The pharmaceutical composition according to any one of claims 93-96, wherein the pharmaceutical composition showed reduced degradation when stored below 5° C.

98. The pharmaceutical composition according to any one of claims 1-97, wherein the pharmaceutical composition has a low bulk density.

99. The pharmaceutical composition according to any one of claims 1-98, wherein the pharmaceutical compositions has been reconstituted into a solution.

100. The pharmaceutical composition of claim 99, wherein the solution is made with water.

101. The pharmaceutical composition of claim 100, wherein the solution is made with phosphate buffered saline.

102. The pharmaceutical composition according to any one of claims 1-101, further comprising one or more additional excipients.

103. The pharmaceutical composition of claim 102, wherein the additional excipient is a protein, an amino acid, a second pharmaceutically acceptable polymer, an antioxidant, or a surfactant.

104. The pharmaceutical composition according to any one of claims 1-103, wherein the pharmaceutical composition comprising:
(A) a nucleic acid; wherein the nucleic acid is an mRNA that encodes for a SARS CoV2 antigen; wherein the SARS CoV2 antigen is a modified version of the SARS CoV2 spike protein;
(B) one or more lipids sufficient to form a lipid nanoparticle; wherein the lipid nanoparticle comprises a first, second, third, and fourth lipid; wherein the first lipid is ionizable lipid, the second lipid is a phospholipid, the third lipid is a PEGylated lipid, and the fourth lipid is a sterol;
(C) a sugar, wherein the sugar is a disaccharide; and
(D) a pharmaceutically acceptable polymer; wherein the pharmaceutically acceptable polymer is a triblock copolymer with two polyoxyethylene units and one polyoxypropylene unit.

105. A method of preparing a pharmaceutical composition according to any one of claims 1-104 comprising:
(A) dissolving a mixture of lipid nanoparticle encapsulating a nucleic acid, a sugar, and a pharmaceutically acceptable polymer in a solvent to obtain a pharmaceutical mixture;
(B) applying the pharmaceutical mixture to a surface at a surface temperature below 0° C. to obtain a frozen pharmaceutical mixture; and
(C) collecting the frozen pharmaceutical mixture and drying the frozen pharmaceutical mixture to obtain a pharmaceutical composition.

106. The method of claim 105, wherein the solvent is water.

107. The method of either claim 105 or claim 106, wherein the pharmaceutical mixture further comprises a second solvent.

108. The method of claim 107, wherein the second solvent is an organic solvent.

109. The method of claim 108, wherein the organic solvent is acetonitrile, tert-butanol, or 1,4-dioxane.

110. The method according to any one of claims 105-109 further comprising admixing the mixture with a salt.

111. The method according to any one of claims 105-110, wherein the first solvent is mixed with the second solvent to obtain a homogenous pharmaceutical mixture.

112. The method according to any one of claims 105-111, wherein the pharmaceutical mixture is admixed until the pharmaceutical mixture is clear.

113. The method according to any one of claims 105-112, wherein the pharmaceutical mixture comprises a solid content from about 0.05% w/v to about 25% w/v of the mixture.

114. The method of claim 113, wherein the solid content is from about 0.1% w/v to about 10% w/v of the mixture.

115. The method of claim 114, wherein the solid content is from about 0.15% w/v to about 5% w/v of the mixture.

116. The method of claim 115, wherein the solid content is from about 0.2% w/v to about 2.5% w/v of the mixture.

117. The method of claim 116, wherein the solid content is from about 0.5% w/v to about 1.25% w/v of the mixture.

118. The method according to any one of claims 105-117, wherein the pharmaceutical mixture is applied at a feed rate from about 0.5 mL/min to about 5 mL/min.

119. The method of claim 118, wherein the feed rate is from about 1 mL/min to about 3 mL/min.

120. The method of claim 119, wherein the feed rate is about 2 mL/min.

121. The method according to any one of claims 105-120, wherein the pharmaceutical mixture is applied with a nozzle.

122. The method of claim 121, wherein the nozzle is a needle.

123. The method according to any one of claims 105-122, wherein the pharmaceutical mixture is applied from a height from about 2 cm to about 50 cm.

124. The method of claim 123, wherein the height is from about 5 cm to about 20 cm.

125. The method of claim 124, wherein the height is about 10 cm.

126. The method according to any one of claims 105-125, wherein the surface temperature is from about 0° C. to −190° C.

127. The method of claim 126, wherein the surface temperature is from about −25° C. to about −125° C.

128. The method of claim 127, wherein the surface temperature is about −100° C.

129. The method according to any one of claims 105-128, wherein the surface is a rotating surface.

130. The method of claim 129, wherein the surface is rotating at a speed from about 5 rpm to about 500 rpm.

131. The method of claim 130, wherein the surface is rotating at a speed from about 100 rpm to about 400 rpm.

132. The method of claim 131, wherein the surface is rotating at a speed of about 200 rpm.

133. The method according to anyone of claims 105-128, wherein the surface is stationary.

134. The method according to any one of claims 105-133, wherein the frozen pharmaceutical composition is dried by lyophilization.

135. The method of claim 134, wherein the frozen pharmaceutical composition is dried at a first reduced pressure.

136. The method of claim 135, wherein the first reduced pressure is from about 10 mTorr to 500 mTorr.

137. The method of claim 136, wherein the first reduced pressure is from about 50 mTorr to about 250 mTorr.

138. The method of claim 137, wherein the first reduced pressure is about 100 mTorr.

139. The method of according to any one of claims 134-138, wherein the frozen pharmaceutical composition is dried at a first reduced temperature.

140. The method of claim 139, wherein the first reduced temperature is from about 0° C. to −100° C.

141. The method of claim 140, wherein the first reduced temperature is from about −20° C. to about −60° C.

142. The method of claim 141, wherein the first reduced temperature is about −40° C.

143. The method according to any one of claims 134-142, wherein the frozen pharmaceutical composition is dried for a primary drying time period from about 3 hours to about 36 hours.

144. The method of claim 143, wherein the primary drying time period is from about 6 hours to about 24 hours.

145. The method of claim 144, wherein the primary drying time period is about 20 hours.

146. The method according to any one of claims 134-145, wherein the frozen pharmaceutical composition is dried a secondary drying time period.

147. The method of claim 146, wherein the frozen pharmaceutical composition is dried a secondary drying time at a second reduced pressure.

148. The method of claim 147, wherein the secondary drying time is at a reduced pressure is from about 10 mTorr to 500 mTorr.

149. The method of claim 148, wherein the secondary drying time is at a reduced pressure is from about 50 mTorr to about 250 mTorr.

150. The method of claim 149, wherein the secondary drying time is at a reduced pressure is about 100 mTorr.

151. The method of according to any one of claims 147-150, wherein the frozen pharmaceutical composition is dried a secondary drying time at a second reduced temperature.

152. The method of claim 151, wherein the second reduced temperature is from about 0° C. to 30° C.

153. The method of claim 152, wherein the second reduced temperature is from about 10° C. to about 30° C.

154. The method of claim 153, wherein the second reduced temperature is about 25° C.

155. The method according to any one of claims 147-154, wherein the frozen pharmaceutical composition is dried for a second time for a second time period from about 0.5 hours to about 36 hours.

156. The method of claim 155, wherein the second time period is from about 6 hours to about 24 hours.

157. The method of claim 156, wherein the second time period is about 20 hours.

158. The method according to any one of claims 134-157, wherein the temperature is changed from the first reduced temperature to the second reduced temperature over a ramping time period.

159. The method of claim 158, wherein the ramping time period is from about 3 hours to about 36 hours.

160. The method of claim 159, wherein the ramping time period is from about 6 hours to about 24 hours.

161. The method of claim 160, wherein the ramping time period is about 20 hours.

162. A pharmaceutical composition prepared using the methods according to any one of claims 105-161.

163. A method of preventing a disease or disorder comprising administering to a patient in need thereof a therapeutically effective amount of a pharmaceutical composition according to any one of claims 1-104 and 162.

164. A method of treating a disease or disorder comprising administering to a patient in need thereof a therapeutically effective amount of a pharmaceutical composition according to any one of claims 1-104 and 162.

165. The method of either claim 163 or claim 164, wherein the disease or disorder is an infection of a virus.

166. The method of claim 166, wherein the virus is SARS CoV2.

167. The method according to any one of claims 163-166, wherein the method reduces the severity of the infection.

168. The method according to any one of claims 163-167, wherein the method prevents the patient from developing a symptomatic infection.
